(19) United States
(12) Patent Application Publication
(10) Pub. No.: US 2026/0148808 A1
(43) Pub. Date: May 28, 2026

(54) SYSTEM AND METHOD FOR OPTIMIZING CHEMICAL REACTIONS USING MACHINE LEARNING

(71) Applicant: MERCK PATENT GMBH, DARMSTADT (DE)

(72) Inventors: MARKO HERMSEN, FRANKFURT AM MAIN (DE); PHILIPP HARBACH, MUEHLTAL (DE); BEN GLASSPOOLE, SHOREWOOD, WI (US); JASMINE GARDNER, MCLEAN, VA (US); THOMAS COLACOT, CHERRY HILL, NJ (US); GUOLIN XU, MENOMONEE FALLS, WI (US)

(21) Appl. No.: 19/121,988

(22) PCT Filed: Oct. 16, 2023
(86) PCT No.: PCT/EP2023/078600

§ 371 Date: Apr. 17, 2025

Related U.S. Application Data
(60) Provisional application No. 63/379,822, filed on Oct. 17, 2022.

(51) Int. Cl. G16C 20/10; G16C 20/70

(52) U.S. Cl. CPC G16C 20/10; G16C 20/70

(57) ABSTRACT

The present disclosure provides methods and systems for optimizing chemical reactions through machine learning. Chemical spaces are defined by grouping prospective chemicals based on selected features. Representative chemicals are selected from each group to assemble a test kit. The test kit is then used to identify the best catalyst or ligand for a catalytic chemical reaction. In some embodiments, the system can recommend a test kit, receive results from experiments, generate a distance matrix, and rank prospective chemicals based on scores obtained for their representative chemicals. The methods involve reducing the dimensionality of chemical features and normalizing distances. The disclosed embodiments can suggest prospective chemicals for optimizing chemical reactions by sorting them based on scores obtained from representative chemicals.

### BACKGROUND

### Relevant Field

The present disclosure relates to optimizing chemical reactions. In particular, the present disclosure relates to a system and method for utilizing a test kit and machine learning for optimizing a catalytic chemical reaction, e.g., by optimizing the selection of one or more catalysts and/or ligands using a design of experiment approach.

### Description of Related Art

Catalysis is a process by which a non-consumable material is added to a chemical reaction to increase speed, efficiency, or otherwise modify the chemical reaction to achieve desired process parameters or results. A catalyst is a substance that speeds up a chemical reaction and/or lowers the activation energy, temperature or pressure needed to start the chemical reaction. Catalysts are not consumed in the reaction and typically remain unchanged after completion of the chemical reaction. A small amount of catalyst is often sufficient to facilitate a chemical reaction.

About 90% of all commercially produced chemical products involve catalysts at some stage in the process of their manufacture. Chemists worldwide in academia and industry regularly optimize catalytic reactions by varying catalytic materials, reactants, solvents, or reaction conditions in order to increase yield, efficiency or cost effectiveness.

In a process chemistry lab, research chemists or lab technicians design synthetic routes to optimize a single catalytic chemical reaction by running several test experiments with approximately 20 catalysts for large scale usage in a manufacturing plant. It may take several weeks to find a suitable catalyst, thereby generating high costs and additional delay in bringing the product to market. In most cases, a catalyst is selected to increase the yield of the reaction and suppress unwanted side-reactions while being safe, cost-effective, and environmentally friendly.

Currently the selection of a catalyst is largely dependent on human intuition and reduced to the most common materials in stock found in a lab or a chemical vendor, thereby ignoring a vast number of other materials within the chemical space, some of which may be more effective than traditional choices. Thus, only a fraction of possible catalysts is typically screened, which do not necessarily lead to an optimal choice.

To comply with and enhance such state of the art it would therefore be desirable to find a new approach to select an optimal catalysis, ligand, or other chemical from among a catalog of many chemicals without having to experimentally try each chemical.

### SUMMARY

According to the present disclosure, a method to assemble a Test Kit for optimizing catalytic chemical reactions via a computer utilizing machine learning is disclosed. The method may include the acts of: Parametrizing the catalysts for the catalytic chemical reactions regarding to respective chemical features which are specific for the catalytic chemical reaction via the computer; Grouping the parametrized catalysts into a given number of clusters, which are spanning over the whole chemical space of the catalysts, based on their chemical featurization and molecular descriptors; Using the computer to select one representative catalyst from every cluster according to specific given criteria; and Assembling the Test Kit with the selected representative catalysts as components comprising.

This approach differs from the known prior art because, inter alia:
1. The incorporation of the physical Test Kit into the workflow with purpose of identifying optimized ligands and catalysts without knowledge of the reaction.
2 The Test Kit may optionally be standardized to support a variety of reactions.
3. The clustering algorithm includes identifying an optimal set of catalysts or ligands for the Test Kit through combination of chemical features and commercial feasibility and/or availability.

Advantageous and therefore exemplary further developments of this disclosure emerge from the associated dependent claims and from the description and the associated drawings.

One of those exemplary further embodiments of the disclosed method comprise that the respective chemical features are determined via cheminformatics and computational modelling on the computer.

Another one of those exemplary further embodiments of the disclosed method comprise that the grouping is performed by the computer via a k-means clustering, a density-based spatial clustering of applications with noise (“DBSCAN”), a spectral clustering, a gaussian mixture model, or other clustering algorithm known to a person of ordinary skill in the relevant art. For example, a density-based, a distribution-base, a centroid-based, or a hierarchical based clustering algorithm may be utilized.

Another exemplary embodiment of the present disclosure selects a chemical based on a combination of chemical features, commercial feasibility, and/or sourcing availability of the catalysts or ligands.

Another exemplary embodiment of the disclosed method comprise storing all available catalysts or ligands in a database connected to a computer.

Another exemplary embodiment uses a Test Kit with a specific given number of catalyst or ligand components assembled by using a method disclosed herein.

Another embodiment of the present disclosure includes a method to optimize a catalytic chemical reaction using the test kit supported by a computer comprising the following optional acts of: Performing standardized experiments for the catalytic chemical reactions with the components in the test kit; Inputting the result data of the performed experiments into the computer; Using a machine learning algorithm, for example, such as but not limited to, a clustering algorithm, a regression algorithm, a categorizing algorithm, an unsupervised machine learning algorithm, a regression model, etc. running on the computer to interpolate between the given number of clusters in the spanned chemical space of all available catalysts or ligands; Using the machine learning regression model to predict the best fitting catalyst for the catalytic chemical reaction in the interpolated parameter space of all available catalysts or ligands; and performing the catalytic chemical reaction with the predicted catalyst or ligand.

One exemplary further embodiment of this disclosed method comprise that a web interface is provided via the computer via which a user uploads the value of yields of the chemical reactions from the performed experiments.

It is understood that all aspects of those preferred further developments can be combined together, even if it is not stated explicitly unless it is obviously impossible due to the nature of the respective features.

A system comprising one or more computers can be configured to perform specific operations or actions through the installation of software, firmware, hardware, or a combination thereof. Such software, firmware, or hardware can, when in operation, cause the system to perform the desired actions. Additionally, one or more computer programs may be designed to carry out specific operations or actions by including instructions that, when executed by data processing apparatus, cause the apparatus to perform the desired actions.

In one general aspect, the method may include selecting a chemical space for grouping. Each chemical space may be defined by a plurality of prospective chemicals. The method may also include selecting a plurality of chemical features, where each chemical feature corresponds to the plurality of prospective chemicals. Furthermore, the method may include grouping the plurality of prospective chemicals in a grouping space based on the plurality of chemical features. In addition, the method may include selecting a plurality of representative chemicals from the prospective chemicals. Each representative chemical corresponds to a group of the plurality of prospective chemicals as grouped within the grouping space. Moreover, the method may include assembling a test kit having a plurality of test chemicals. Each test chemical corresponds to a representative chemical of the plurality of representative chemicals. Other embodiments of this aspect include corresponding computer systems, apparatus, and computer programs recorded on one or more computer storage devices. Each may be configured to perform the actions of the methods.

In one general aspect, the method may include implementations that may include one or more of the following features as part of a method. A method may include where the grouping space is defined by the plurality of chemical features. The grouping space may be a dimensionally reduced space of the plurality of chemical features. The method may also include the act of generating a plurality of reduced chemical spaces where each reduced chemical spaces is a dimensionally reduced space of the chemical space. The method may include calculating a plurality of distances where each distance of the plurality of distances is a distance between a prospective chemical of the plurality of prospective chemicals and a test chemical of the plurality of test chemicals. The plurality of test chemicals may be a subset of the plurality of prospective chemicals. The plurality of prospective chemicals may not include the plurality of test chemicals. The method may include calculating a plurality of distance metrics where each distance metric of the plurality of distance metrics is one minus a distance between one of the plurality of prospective chemicals and one of the plurality of test chemicals.

In one general aspect, the method may include implementations that may include one or more of the following features as part of a method. The method may include averaging each of the plurality of distance metrics across all of the reduced chemical spaces to generate a plurality of averaged distance metrics. The method may include generating a plurality of weights where each weight of the plurality of weights corresponds to one of a plurality of results and where each result of the plurality of results corresponds to one of the plurality of test chemicals. The plurality of weights may be determined in accordance with an exponential function. Each of a plurality of distances of distance metrics may be multiplied by a respective weight of the plurality of weights. A maximum value for each respective prospective chemical may be taken across all of the multiplied plurality of distance metrics between the respective prospective chemical and the plurality of test chemicals. The method may include ranking the maximum value for each respective prospective chemical. The method may include testing the plurality of test chemicals to determine a plurality of results where each of the plurality of results corresponds to a respective result of a test chemical of the plurality of test chemicals.

In one general aspect, the method may include implementations that may include one or more of the following features as part of a method. The plurality of results may be uploaded into a server. The method may include selecting a prediction space utilizing a plurality of predictive features to provide a prediction to a result space where the plurality of results define data points within the prediction space mapped to the result space. The result space may include a chemical yield. The result space may be a side-product metric. The result space may be a single parameter. The prediction space may include the grouping space. The method may include selecting a chemical from the plurality of prospective chemicals corresponding to an optimized value of the result space. The prediction space can include the plurality of chemical features therein in some aspects thereof. A computer may be configured to fit the plurality of results using a regression fit to map the plurality of predictive features to the result space. The computer may be configured to train a learner using the plurality of results.

In one general aspect, the method may include implementations that may include one or more of the following features as part of a method. The learner may include at least one of an artificial neural network, a K-nearest neighbor, a decision tree, a random forest, a support vector machine, a Bayesian regressor, and/or an ensemble. The plurality of predictive features may include at least one of a space-filling feature, a bulk feature, an orientation feature, an electrical feature, a Vin, a frontier Mos, a Fukui function, an NBO analyses, a NMR tensor, a steric property, a Sterimol L, B1, B5, B1, B5, a quadrant analysis, an octant analysis, a total volume, a buried volume, a dipole moment, an energy of solvation, a dispersion of potential, etc. The plurality of chemical features may include a plurality of chemical featurizations. The plurality of chemical features may include a plurality of molecular descriptors. The chemical space may include a plurality of catalysts. The chemical space may include a plurality of ligands. The plurality of chemical features may be determined via at least one of cheminformatics and computational modelling on the computer. The plurality of chemical features may be stored on a database executed by a computer.

In one general aspect, the method may include implementations that may include one or more of the following features as part of a method. The method may include testing the plurality of test chemicals to determine a plurality of results where each of the plurality of results corresponds to a respective result of a test chemical of the plurality of test chemicals; selecting a prediction space utilizing a plurality of predictive features to provide a prediction to a result space where the plurality of results define data points within the prediction space; mapping the plurality of predictive features to the result space using regression; determining a best catalyst or best ligand of all available catalysts or ligands for a catalytic chemical reaction in the prediction space in accordance with a prediction; and/or performing the catalytic chemical reaction with the best catalyst or best ligand in accordance with the prediction.

In one general aspect, the method may include implementations that may include one or more of the following features as part of a method. The plurality of results may be uploaded into a server. The method may include selecting the prediction space utilizing a plurality of predictive features to provide a prediction to a result space where the plurality of results defines data points within the prediction space. The computer may be configured map the plurality of predictive features to the result space using regression by fitting the plurality of results to the predictive features and the result space. The grouping space may be generated by performing dimensionality reduction on the plurality of chemical features. The dimensionality reduction may be implemented on a computer utilizing principal component analysis. The chemical space may be defined by a plurality of phosphine ligands for cross-coupling reactions. The cross-coupling reactions may include one of a Suzuki catalysis and a Buchwald catalysis. The plurality of test chemicals may include 24 chemicals. The plurality of chemical features may include at least one of a space-filling feature, a bulk feature, an orientation feature, an electrical feature, a Vin, a frontier Mos, a Fukui function, an NBO analyses, a NMR tensor, a steric property, a Sterimol L, B1, B5, B1, B5, a quadrant analysis, an octant analysis, a total volume, a buried volume, a dipole moment, an energy of solvation, and/or a dispersion of potential. Implementations of the described techniques may include hardware, a method or process, or a computer tangible medium.

In one general aspect, a method may include assembling a test kit having a plurality of test chemicals where each test chemical corresponds to a representative chemical of a plurality of representative chemicals; testing the plurality of test chemicals to determine a plurality of results where each of the plurality of results corresponds to a respective result of a respective test chemical of the plurality of test chemicals; and determining a best catalyst or best ligand of all available catalysts or ligands for a catalytic chemical reaction in a prediction space in accordance with a prediction. Other embodiments of this aspect include corresponding computer systems, apparatus, and computer programs recorded on one or more computer storage devices, each configured to perform the actions of the methods.

Implementations may include one or more of the following features. The method may include: generating a plurality of reduced chemical spaces where each reduced chemical spaces is a dimensionally reduced space of a chemical space; calculating a plurality of distances where each distance of the plurality of distances is a distance between a prospective chemical of a plurality of prospective chemicals and a test chemical of the plurality of test chemicals; calculating a plurality of distance metrics where each distance metric of the plurality of distance metrics is one minus a distance between one of the plurality of prospective chemicals and one of the plurality of test chemicals; and/or averaging each of the plurality of distance metrics across all of the reduced chemical spaces to generate a plurality of averaged distance metrics.

In one general aspect, the method may include implementations that may include one or more of the following features as part of a method. The method may include generating a plurality of weights where each weight of the plurality of weights corresponds to one of a plurality of results and where each result of the plurality of results corresponds to one of the plurality of test chemicals. The plurality of weights may be determined in accordance with an exponential function. Each of a plurality of distances of distance metrics may be multiplied by a respective weight of the plurality of weights. A maximum value for each respective prospective chemical may be taken across all of the multiplied plurality of distance metrics between the respective prospective chemical and the plurality of test chemicals.

In one general aspect, the method may include implementations that may include one or more of the following features as part of a method. The method may include ranking the maximum value for each respective prospective chemical to thereby find the best catalyst or best ligand; selecting the prediction space utilizing a plurality of predictive features to provide a prediction to a result space where the plurality of results define data points within the prediction space; and/or fitting the data points within the plurality of predictive features to the result space using regression. The plurality of predictive features may include at least one of a space-filling feature, a bulk feature, an orientation feature, an electrical feature, a Vin, a frontier Mos, a Fukui function, an NBO analyses, a NMR tensor, a steric property, a Sterimol L, B1, B5, B1, B5, a quadrant analysis, an octant analysis, a total volume, a buried volume, a dipole moment, an energy of solvation, a dispersion of potential, etc. The method may include performing the catalytic chemical reaction with the best catalyst or best ligand in accordance with the prediction. Implementations of the described techniques may include hardware, a method or process, or a computer tangible medium.

In one general aspect, a method may include parametrizing catalysts or ligands for a respective catalytic chemical reaction regarding respective chemical features which are specific for the catalytic chemical reaction via the computer. The method may also include grouping the parametrized catalysts or ligands into a given number of clusters which are spanning over a chemical space of the catalysts or ligands, based on their chemical features. Furthermore, the method may include using the computer to select one representative catalyst or ligand from each cluster according to predetermined criteria. The method may, in addition, include assembling the Test Kit with the selected representative catalysts or ligands as components. Other embodiments of this aspect include corresponding computer systems, apparatus, and computer programs recorded on one or more computer storage devices, each configured to perform the actions of the methods.

Implementations may include one or more of the following features. The respective chemical features may be determined via cheminformatics and computational modelling on the computer. The method may perform the grouping act by using k-means clustering using the computer. A selection may be based on a combination of chemical features and commercial feasibility and/or sourcing availability of the catalysts or ligands as the predetermined criteria. All available catalysts and/or ligands may be stored on a database connected to the computer.

In one general aspect, the method may include implementations that may include one or more of the following features as part of a method. The method may include: performing standardized experiments for the catalytic chemical reactions with the components in the Test Kit; inputting result data of the performed experiments into the computer; using a machine learning regression model running on the computer to interpolate between the given number of clusters in the spanned chemical space of all available catalysts or ligands; using the machine learning regression model to predict the best fitting catalyst for the catalytic chemical reaction in the spanned chemical space of all available catalysts or ligands; and/or performing the catalytic chemical reaction with the predicted catalyst or ligand. A web interface may be provided via the computer via which an user uploads the results data of the chemical reactions from the performed experiments. Implementations of the described techniques may include hardware, a method or process, or a computer tangible medium.

In one general aspect, a system disclosed herein may select a chemical space for grouping where each chemical space defined by a plurality of prospective chemicals; select a plurality of chemical features where each chemical feature corresponding to the plurality of prospective chemicals, group the plurality of prospective chemicals in a grouping space based upon the plurality of chemical features; select a plurality of representative chemicals from the prospective chemicals where each representative chemical corresponding to a group of the plurality of prospective chemicals as grouped within the grouping space; and/or recommend a test kit having a plurality of test chemicals where each test chemical corresponding to a representative chemical of the plurality of representative chemicals. Other embodiments of this aspect include corresponding computer systems, apparatus, and computer programs recorded on one or more computer storage devices, each configured to perform the actions of the methods.

In one general aspect, a disclosed system may receive a plurality of results from a plurality of experiments performed using the plurality of test chemicals where each of the plurality of results corresponds to a respective result of a respective test chemical of the plurality of test chemicals. The system may also recommend a best catalyst or best ligand of all available catalysts or ligands for a catalytic chemical reaction in a prediction space in accordance with a prediction. Other embodiments of this aspect include corresponding computer systems, apparatus, and computer programs recorded on one or more computer storage devices, each configured to perform the actions of the methods.

In one general aspect, the method may include performing an initial screening experiment on a set of test chemicals to generate results. The method may also include obtaining chemical features that describe properties of the prospective chemicals. The method may furthermore include reducing a dimensionality of the chemical features to generate a predetermined number of different chemical spaces. The method may, in addition, include calculating distances between each of the predetermined number of chemical spaces and each of the test chemicals for a given prospective chemical. The method may moreover include normalizing the distances for each of the predetermined number of chemical spaces to the [0,1] interval. The method may also include subtracting the normalized distances from 1 to generate a distance metric for each prospective chemical and each test chemical in each chemical space.

In one general aspect, the method may include implementations that may include one or more of the following features as part of a method. The method may furthermore include averaging the distance metrics over all chemical spaces for each prospective chemical. The method may in addition include normalizing the results obtained from the initial screening experiment to [0,1] and converting them into weights. The method may moreover include multiplying the weights with a distance matrix to obtain a weighted distance matrix, the distance matrix generated via the averaging act and having a prospective chemical axis and a test chemical axis. The method may also include taking the maximum value along the prospective chemical axis to obtain a score for each prospective chemical. The method may furthermore include ranking the N prospective chemicals from highest to lowest based on the obtained scores. Other embodiments of this aspect include corresponding computer systems, apparatus, and computer programs recorded on one or more computer storage devices, each configured to perform the actions of the methods.

Implementations may include one or more of the following features. The chemical features may be DFT-based features. The chemical features may be reduced using PCA, Space PCA, Kernel PCA with an RBF kernel, Kernel PCA with a cosine kernel, Fast ICA, Spectral Embedding, Isomap, or Local Linear Embedding. The results obtained from the initial screening experiment may be yield or enantioselectivity. The weights obtained from the normalized results are column-wise multiplied with the distance matrix to obtain a weighted distance matrix. The chemical features are phosphine ligands' properties. Implementations of the described techniques may include hardware, a method or process, or a computer tangible medium.

In one general aspect, the method may include: obtaining a list of N prospective chemicals; obtaining chemical features configured to describe properties of the N prospective chemicals; clustering the N prospective chemicals based on their chemical features to obtain a set of representative chemicals, the set of representative chemicals define a set of test chemicals; calculating distances between each of the test chemicals and each of the representative chemicals using a distance metric based on the chemical features; normalizing the distances to the [0,1] interval; subtracting the normalized distances from 1 to generate a distance metric for each representative chemical and each test chemical; normalizing results obtained from an initial screening experiment to [0,1] and converting them into weights; multiplying the weights with a distance matrix to obtain a weighted distance matrix; taking the maximum value along a representative chemical axis to obtain a score for each representative chemical; and/or ranking the N prospective chemicals based on the scores obtained for their representative chemicals. Other embodiments of this aspect include corresponding computer systems, apparatus, and computer programs recorded on one or more computer storage devices, each configured to perform the actions of the methods.

Implementations may include one or more of the following features. A computer program product may be implemented as a system or method described herein (as a computer-readable medium and/or a non-transitory computer-readable medium, etc). In some embodiments, the distances may be calculated after dimensionality reduction of the chemical features. Implementations of the described techniques may include hardware, a method or process, or a computer tangible medium.

In one general aspect, the method may include obtaining a list of N prospective chemicals. Method may also include receiving results for M prospective chemicals thereby defining test chemicals; obtaining chemical features configured to describe properties of the N prospective chemicals; determining a score for each representative chemical; and ranking the N prospective chemicals based on the scores obtained for their representative chemicals. Other embodiments of this aspect include corresponding computer systems, apparatus, and computer programs recorded on one or more computer storage devices, each configured to perform the actions of the methods.

Implementations may include one or more of the following features. The act of determining the score for each representative chemical may include: calculating distances between each of the test chemicals and each of the representative chemicals using a distance metric for each of a plurality of dimensionally reduced spaces of the chemical features; normalizing the distances to the [0,1] interval for each of the plurality of dimensionally reduced spaces; subtracting the normalized distances from 1 to generate a distance metric for each representative chemical and each test chemical for each of the plurality of dimensionally reduced spaces; averaging the distance metric for each representative chemical and each test chemical across all of the dimensionally reduced spaces; normalizing the results obtained from the received results of the M prospective chemicals to [0,1] and converting them into weights; multiplying the weights with a distance matrix to obtain a weighted distance matrix, the distance matrix having a representative chemical axis and a test chemical axis; and/or taking the maximum value along a representative chemical axis to obtain a score for each representative chemical. The test chemicals may be removed from the representative chemical axis. Implementations of the described techniques may include hardware, a method or process, or a computer tangible medium.

### BRIEF DESCRIPTION OF THE DRAWINGS

These and other aspects will become more apparent from the following detailed description of the various embodiments of the present disclosure with reference to the drawings wherein:

FIG. 1 shows a schematic overview of a system for optimizing chemical reactions using machine learning in accordance with an embodiment of the present disclosure;

FIG. 2 illustrates clustering of phosphine ligands in accordance with an embodiment of the present disclosure;

FIG. 3 illustrates clustering of phosphine ligands based on molecular featurization and sets of molecular descriptors in accordance with an embodiment of the present disclosure;

FIG. 4 illustrates the use of a Physical kit containing 24 phosphine ligands from each of the clusters representing the whole chemical space in accordance with an embodiment of the present disclosure;

FIG. 5 illustrates an Artificial Intelligence-based web platform to suggest one or more optimal catalysts for use in a reaction with a specific set of reactants in accordance with an embodiment of the present disclosure; and

FIG. 6 shows a system for selecting one or more optimal chemicals in accordance with an embodiment of the present disclosure.

### DETAILED DESCRIPTION

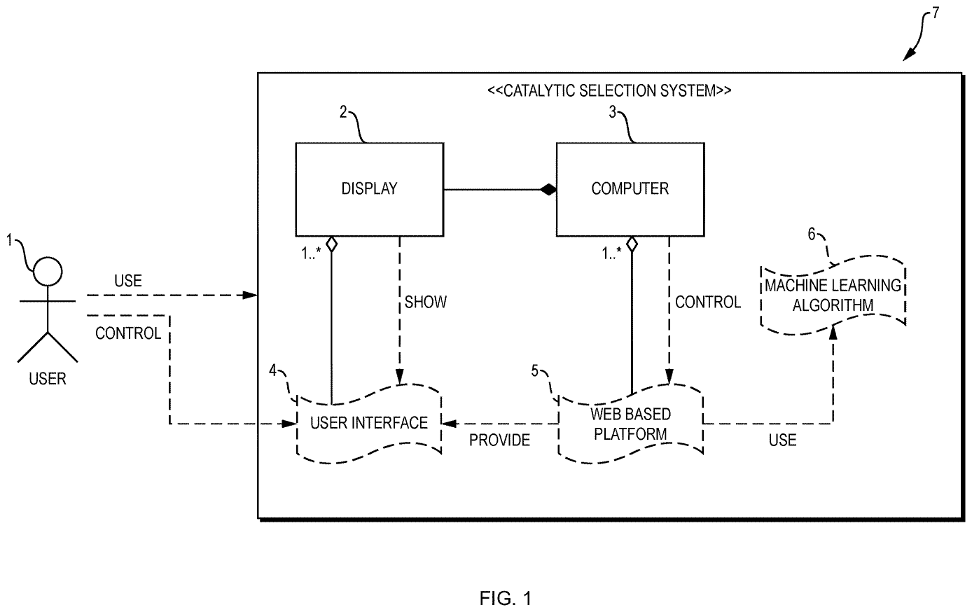

FIG. 1 shows a schematic overview of a system 7 to use the assembled Test Kit to find an optimal catalyst for the desired catalytic chemical reaction. The catalyst selection system 7 comprises of a web-based platform 5, e.g. realized as a homepage, which runs on a computer 3 in form of a server and which is accessible by a user 1 via a web browser on a remote device, just like a mobile phone, a personal computer or the like. The web-based platform 5 provides therefore a user interface 4, preferably a graphical user interface (GUI) through which the user 1 can input the results of the experiments he performed by using the assembled Test Kit. Furthermore, the web-based platform can also be configured to provide via the homepage the functionality to assemble the Test Kit in the first place according to information given by the user 1, for example, the desired catalytic chemical reaction. With this information and further data about the availability of specific catalysts or ligands, the system 7 can compute the Test Kit components and submit a request for assembly of the Test Kit.

For example, in one embodiment of the present disclosure, a “Phosphine Predictor” suggests phosphine ligands for cross coupling reactions, e.g. Suzuki and Buchwald catalysis. It suggests which commercially available monodentate phosphine ligand should be tried for the C—C or C—N cross coupling reaction of specific reaction substrates. The user runs a reaction with the offered designated Universal Training Set ligands (the Test Kit) and inputs the results (which can be the yields, efficiencies, or cost effectiveness) into the secure Phosphine Predictor portal (e.g., vis the user interface 4). Optimal ligand suggestions will be uploaded to a user account.

The system 7 also comprises a machine learning algorithm 6 that is described herein. The machine learning algorithm 6 may be designed to learn patterns and make predictions or decisions based on data it is given. The machine learning algorithm 6 may use a dataset of various chemical as described herein. The model can be used to make predictions for hypothetical chemical reactions. For a new input, the machine learning algorithm may make prediction based on the patterns it learned previously or using any algorithm as described herein. The system 7 may utilize CPUs and/or GPUs to parallelize computations on the computer 3.

This exemplary embodiment will be further introduced by describing method acts which further explains the specific example of the disclosed method. The method in one embodiment is included in an AI-based Design of Experiment platform. The embodiment is not limited to the hereby disclosed and used hardware.

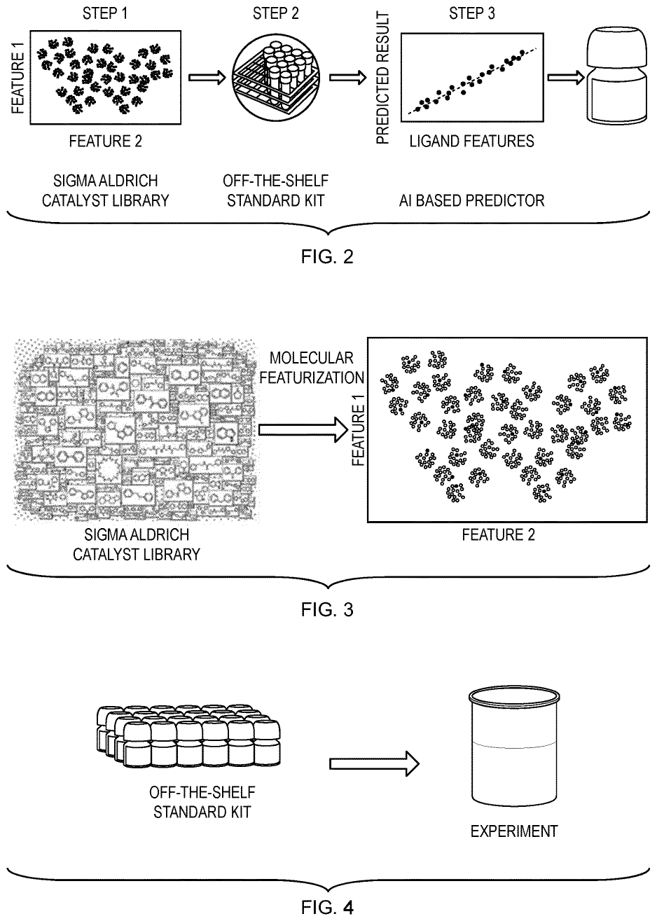

FIG. 2 shows an overview of all three steps of a method of the present disclosure: The clustering of phosphine ligands based on molecular featurization and sets of molecular descriptors (Step 1), a Physical kit containing 24 phosphine ligands from each of the clusters (step 2), and an AI based web platform to suggest the best catalysts for a reaction with specific sets of reactants (step 3).

FIG. 3 shows then the clustering of phosphine ligands via a k-means clustering approach to screen all ligands available on the respective used web platform or homepage. The catalysts/ligands are parameterized with chemical features coming from cheminformatics and computational modelling spanning the chemical space. The catalysts or ligands are then grouped into 24 clusters based on the chemical featurization and molecular descriptors.

In FIG. 4, one representative catalysts/ligand from each of 24 clusters may be included in a physical kit which will be available for purchase via the web-based platform 5. The selection is based on sourcing availability. A potential customer acquires the kit and performs test experiments with all 24 representative catalysts/ligands from the physical kit for a specific catalytic chemical reaction of their interest keeping all other reaction condition as constant if possible.

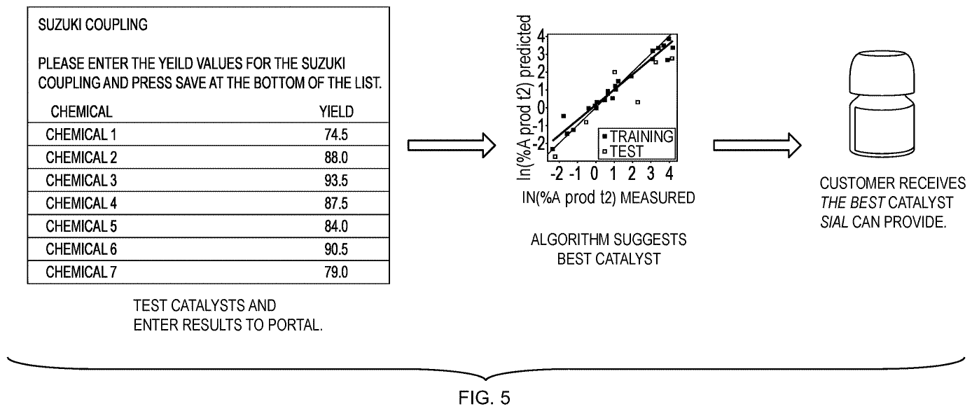

The customer inputs the value of yields of the 24 chemical reactions into a web portal or interface of the web based platform 5 as can be seen in FIG. 5. Based on the input the system performs a regression to interpolate between the 24 clusters in the chemical space of the catalysts/ligands and will recommend the best possible catalysts/ligands for the given reaction.

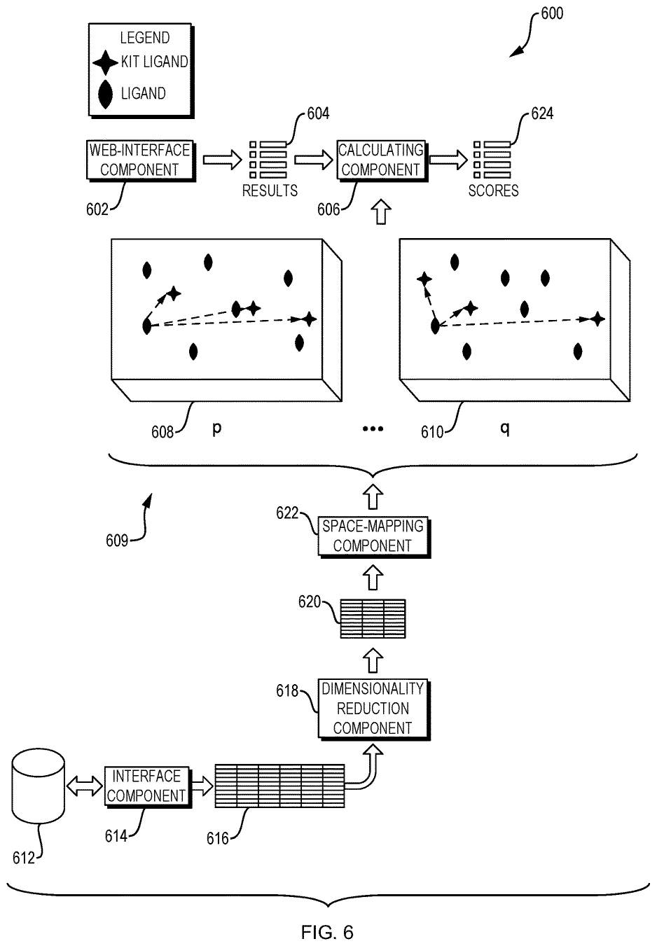

FIG. 6 shows a system 600 for suggesting prospective chemicals that could potentially optimize chemical reactions based on a combination of distance metrics and performance results. The system 600 may be implemented on the computer 3 of FIG. 1. The system 600 may be implemented in hardware, software, software being executed by a processor and/or GPU etc. In some embodiments, the system 600 may be implemented in the cloud, as a software-as-a-service platform, as a distributed system, etc. The system 600 includes an interface component 614 that retrieves chemical features 616 from a database 612 and a web-interface component 602 that receives the results 604 from the test chemicals uploaded by a user.

The interface component 614 obtains chemical features 616 that describe the properties of a given set of prospective chemicals. These chemical features 616 can be obtained using standard interfaces, such as web interfaces, REST API, etc. In one specific embodiment, the chemical features used were DFT-based features as described in Gensch, T.; dos Passos Gomes, G.; Friederich, P.; Peters, E.; Gaudin, T.; Pollice, R.; Jorner, K.; Nigam, A.; Lindner-D'Addario, M.; Sigman, M. S.; Aspuru-Guzik, A.; A Comprehensive Discovery Platform for Organophosphorus Ligands for Catalysis. J. Am. Chem. Soc. 2022, 144, 3, 1205-1217, the contents of which is incorporated herein by reference in its entirety. However, any other type of chemical feature that describes the properties of prospective chemicals can also be used. For example, properties of phosphine ligands relevant for optimizing chemical reactions utilizing these chemicals (such as catalysts) can also be considered.

Due to the high dimensionality of the chemical features 616, dimensionality reduction is applied to the chemical features by a dimensionality reduction component 618. In a specific embodiment of the present disclosure, dimensionality reduction is applied nine times using different techniques to generate nine different chemical spaces 609. For ease of viewing, two chemical spaces 608, 610 are shown as spanning from space p to space q. Any suitable number of chemical spaces 609 may be used. As an alternative embodiment, other dimensionality reduction techniques such as autoencoders and t-SNE can also be employed. The aim of dimensionality reduction is to reduce the number of features to a manageable number that can be efficiently and effectively used to compare the properties of prospective chemicals.

As mentioned, the chemical features 616 are reduced to nine different embedding 620 that can be mapped using the space-mapping component 622 to nine different chemical spaces 609. The dimensionality reductions may be done using PCA, Space PCA, Kernel PCA with an RBF kernel, Kernel PCA with a cosine kernel, Fast ICA, Spectral Embedding, a Isomap, Local Linear Embedding and multidimensional scaling. Each of the embeddings 620 may be 20 dimensions in some specific embodiments.

Once the chemical features 616 have been reduced and mapped via the space-mapping component 622, the distances to a given set of test chemicals are calculated for each prospective chemical within each of the generated chemical spaces 609. The distance is computed using reduced chemical features that describe the properties of prospective chemicals relevant for optimizing chemical reactions. If there are 24 test chemicals and nine spaces 609, then 24 times 9 distances are calculated, resulting in a total of 216 distances for each prospective chemical.

The distances for each of the nine chemical spaces are then normalized to the [0,1] interval (e.g., Euclidean distances). The normalizations may be per space of the spaces 609 (e.g., because the absolute value is highly dependent on that space). For example, in specific embodiments, there may be 9 sets of M×N distances where each set of the M×N distances may be normalized to 0,1. Any normalization may be used, such a linear normalization, min-max normalizations, etc. This allows for a standardization of the distances and for a more easily comparable analysis of the results 604. Subsequently, all distances are subtracted from 1 to generate a distance metric where the shortest distance is now 1 and the greatest distance is 0 within each chemical space. As an alternative embodiment, other types of distance metrics such as Mahalanobis distance, Manhattan distance, Euclidean distance etc. can also be used. The choice of distance metric depends on the nature of the chemical properties being compared and the specific application. Additionally, alternatively, or optionally, any post-normalization techniques such as rescaling or standardizing of the distances can also be employed.

Next, the mean over all distance metrics with respect to the different embeddings (i.e., chemical spaces 609) for each prospective chemical is taken. This generates a matrix of N×M, where N is the number of prospective chemicals for which recommendations are made and M is the number of test chemicals (e.g., M=24). Each location within the matrix may represent the average (mean) of the nine distance metrics across all spaces 609 for a specific prospective chemical to a specific test chemical. As an alternative embodiment, this step can be performed using other mean calculation techniques, such as weighted-average mean or geometric mean.

The results 604 from the experiments conducted using the test chemicals may be uploaded by the web-interface component 602. The results 604 (e.g., yield) are also normalized to [0,1] and converted into weights using an exponential function of the form 2\hat{ }(x−1), where x is the result. These weights are then multiplied with the distance matrix column-wise. The maximum value along the prospective chemical axis is taken, resulting in a score ranging from 0 to 1 for each prospective chemical, representing the best distance/performance combination with respect to the test chemicals. These values are considered scores 624. As an alternative embodiment, different methods of score calculation such as weighted sum or product of the distance and performance scores can also be employed.

These scores 624 are then used to rank the N prospective chemicals from highest to lowest, where the higher the value, the better the result is predicted. The disclosed method provides a reliable and efficient way to suggest prospective chemicals that could potentially optimize chemical reactions. By taking the top-scoring prospective chemicals, another set of test chemicals may be proposed and the process can be repeated.

As further alternative embodiments, the disclosed method can be employed in various industries, including pharmaceuticals, materials science, and agrochemicals. The method can also be used to predict the activity of other chemical compounds beyond prospective chemicals, such as drug candidates or natural products. These chemical features can be obtained using various sources, including in silico or in vitro assays. Furthermore, in addition to the nine different dimensionality reduction techniques used in the present disclosure, other techniques such as UMAP or non-negative matrix factorization can also be employed.

Moreover, an alternative embodiment of the distance metric calculation step is the use of machine learning models such as regression models or neural networks. These models can predict the distances between the prospective chemicals and the test chemicals based on the chemical features. The prediction accuracy of these models can be evaluated using cross-validation or testing on holdout data.

Another alternative embodiment of the score calculation step is the incorporation of uncertainty measures such as confidence intervals or probability distributions of the scores. These measures can provide additional information on the reliability and robustness of the score predictions.

In conclusion, the disclosed method and system provides a versatile and comprehensive approach for predicting the most promising prospective chemicals for optimizing chemical reactions. The method allows for the use of various chemical features and dimensionality reduction techniques, as well as alternative distance metric and score calculation methods. These embodiments make the method applicable to various chemical industries and can improve the accuracy and reliability of the predictions.

The present invention is further illustrated by the examples following hereinafter which shall in no way be construed as limiting. A skilled person will acknowledge that various modifications, additions and alternations may be made to the invention without departing from the spirit and scope of the invention as defined in the appended claims.

### EXAMPLES

### General Information

All reagents and solvents were purchased from MilliporeSigma, unless otherwise noted, and used as received. 2′-Dicyclohexylphosphino-2-methoxy-1-phenylnaphthalene, cBRIDP, and di-tert-butyl(2′,6′-dimethoxy-[1,1′-biphenyl]-2-yl)phosphine were purchased from Ambeed. VPhos, tri(m-tolyl)phosphine, 9-[2-(dicyclohexylphosphino)phenyl]-9H-carbazole, CPhos, trioctylphosphine, and bis(3,5-bis(trifluoromethyl)phenyl)(2′,6′-bis(isopropoxy)-3,6-dimethoxybiphenyl-2-yl)phosphine were purchased from STREM. 3-(Diphenylphosphino)phenol and 2-(Dicyclohexylphosphino)-2′-methoxybiphenyl were purchased from Combi-Blocks. 2-Diphenylphosphino-6-methylpyridine and tris(diethylamino)phosphine were purchased from TCI.

Flash chromatography was performed on either a Biotage Isolera™ or Biotage Selekt system. Compounds were characterized by ¹H NMR and ¹³C NMR. NMR spectra were recorded either in a Varian 500 MHz instrument or in a Bruker 500 MHz instrument. All ¹H NMR experiments are reported in 0 units, parts per million (ppm), and were measured relative to the signals for residual chloroform-d (7.26 ppm) and all ¹³C NMR spectra are reported in ppm relative to chloroform-d (77.23 ppm) and all were obtained with ¹H decoupling. All GC analyses were performed on an Agilent 7820A gas chromatograph with an FID detector using a SPB-1 fused silica column 30 m×250 μm×1 um (cat #: 24029). All reaction vials were prepared in a positive pressure Vac Omni-Lab glovebox and reacted on the Radley's Mya4 Reaction Station under positive nitrogen pressure. CAUTION! Neat phosphines can be pyrophoric as they may react with air and moisture; however, pyrophoricity can be minimized when used as a solution. Follow all precautions in the SDS.

### Phosphine Kit Ligands

TABLE S1
24 Representative Phosphine Predictor Kit Ligands and Structures.

| Ligand # | Ligand Name | Ligand Structure |
|---|---|---|
| L1 | Trioctylphosphine | 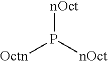 |
| L2 | Methyldiphenylphosphine | 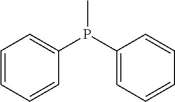 |
| L3 | Tris(diethylamino)phosphine | 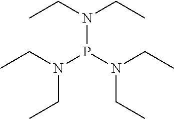 [SMILES: CCN(CC)P(N(CC)CC)N(CC)CC] |
| L4 | Tri(o-tolyl)phosphine | 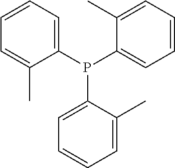 [SMILES: Cc1ccccc1P(c1ccccc1C)c1ccccc1C] |
| L5 | Tri(p-tolyl)phosphine | 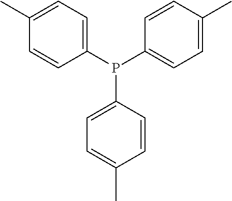 |
| L6 | Tris(pentafluorophenyl)phosphine | 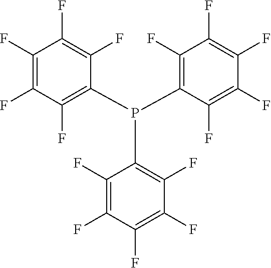 [SMILES: Fc1c(F)c(F)c(P(c2c(F)c(F)c(F)c(F)c2F)c2c(F)c(F)c(F)c(F)c2F)c(F)c1F] |
| L7 | Tripropylphosphine | 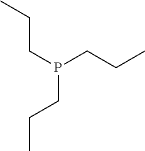 |
| L8 | Diphenyl-2-pyridylphosphine | 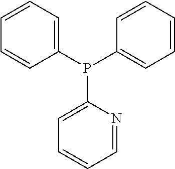 [SMILES: c1ccc(P(c2ccccc2)c2ccccn2)cc1] |
| L9 | Tris(dimethylamino)phosphine | 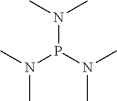 |
| L10 | Tris(2,4,6- trimethylphenyl)phosphine | 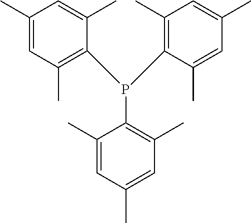 |
| L11 | Tris(4-methoxyphenyl)phosphine | 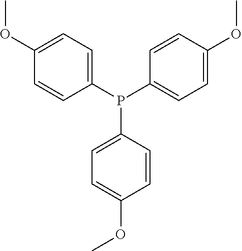 [SMILES: COc1ccc(P(c2ccc(OC)cc2)c2ccc(OC)cc2)cc1] |
| L12 | DavePhos | 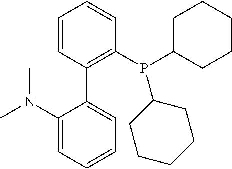 [SMILES: CN(C)c1ccccc1-c1ccccc1P(C1CCCCC1)C1CCCCC1] |
| L13 | Xphos | 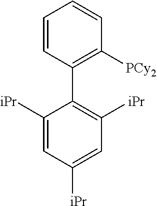 |
| L14 | JohnPhos | 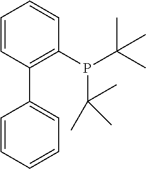 [SMILES: CC(C)(C)P(c1ccccc1-c1ccccc1)C(C)(C)C] |
| L15 | (3aR,8aR)-(−)-(2,2-Dimethyl- 4,4,8,8-tetraphenyl-tetrahydro- [1,3]dioxolo[4,5- e][1,3,2]dioxaphosphepin-6- yl)dimethylamine | 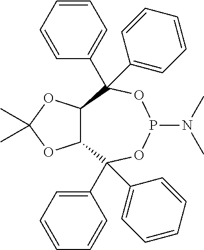 |
| L16 | 2-(Di-tert-butylphosphino)-1- phenylindole | 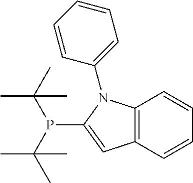 [SMILES: CC(C)(C)P(c1cc2ccccc2n1-c1ccccc1)C(C)(C)C] |
| L17 | APhos | 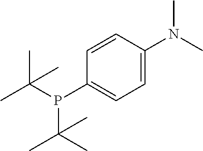 [SMILES: CN(C)c1ccc(P(C(C)(C)C)C(C)(C)C)cc1] |
| L18 | tBuBrettPhos | 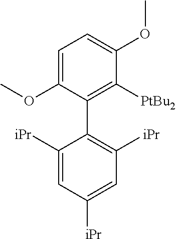 |
| L19 | Bis[2-(trimethylsilyl)ethyl] N,N- diisopropylphosphoramidite | 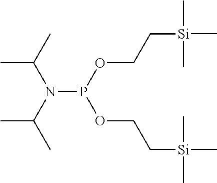 [SMILES: CC(C)N(C(C)C)P(OCC[Si](C)(C)C)OCC[Si](C)(C)C] |
| L20 | Exo-4-anisole Kwon [2.2.1] bicyclic phosphine | 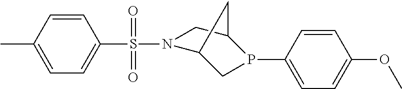 |
| L21 | HandaPhos | 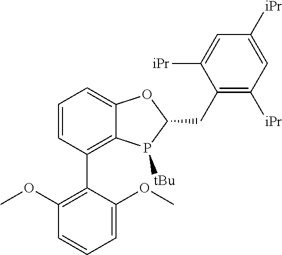 [SMILES: COc1cccc(OC)c1-c1cccc2c1[P@](C(C)(C)C)[C@@H](Cc1c(C(C)C)cc(C(C)C)cc1C(C)C)O2] |
| L22 | Bis(3,5-bis(trifluoromethyl)phenyl) (2′,6′-bis(isopropoxy)-3,6- dimethoxybiphenyl-2- yl)phosphine | 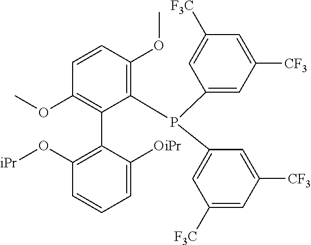 [SMILES: COc1ccc(OC)c(P(c2cc(C(F)(F)F)cc(C(F)(F)F)c2)c2cc(C(F)(F)F)cc(C(F)(F)F)c2)c1-c1c(OC(C)C)cccc1OC(C)C] |
| L23 | Triethyl phosphite | 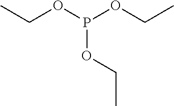 |
| L24 | Triphenyl phosphite | 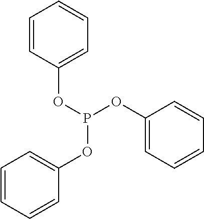 [SMILES: c1ccc(OP(Oc2ccccc2)Oc2ccccc2)cc1] |

### Ligand Prediction Model

The following describes a specific embodiment of the system 600 described above with reference to FIG. 6. For the purpose of understanding how the ligand prediction/recommendation model works as is described herein, especially with reference to FIG. 6, we'll use the terms “kit ligand” (a specific embodiment of a test chemical) for any ligand that is in the set of 24 ligands for which the initial screening experiment is performed, and just “ligand” for any ligand that is not part of this set for which we obtain a ranking to through our model to make suggestions for new experiments (a specific embodiment of prospective chemical).

The model (e.g., system 600) suggests ligands based on two criteria, distance to a kit ligand (how similar to a kit ligand is a ligand) and performance (in our case yield/conversion but could be enantioselectivity or any other criteria) of that kit ligand.

Distances are obtained from molecular features/descriptors that describe the properties of ligands. In our case, we used the DFT-based features as described in the kraken paper (Gensch, T.; dos Passos Gomes, G.; Friederich, P.; Peters, E.; Gaudin, T.; Pollice, R.; Jorner, K.; Nigam, A.; Lindner-D'Addario, M.; Sigman, M. S.; Aspuru-Guzik, A.; A Comprehensive Discovery Platform for Organophosphorus Ligands for Catalysis. J. Am. Chem. Soc. 2022, 144, 3, 1205-1217). These features are available from the website (https://kraken.cs.toronto.edu/) using the REST API or can be computed following the published code as in the corresponding GitHub repository (https://github.com/aspuru-guzik-group/kraken). In principle, any other set of descriptors can be used that describes the phosphine ligands' properties which are relevant for catalysis. Due to the high dimensionality of the features, dimensionality reduction was applied to yield embeddings (e.g., embedding 620) with 20 components. Again, various techniques may be used for this-we used in total nine dimensionality reduction methods (PCA, Sparse PCA, Kernel PCA with an RBF kernel and with a cosine kernel, Fast ICA, Spectral Embedding, Isomap, Locally Linear Embedding and MDS). Within each of these embeddings, the distance of each ligand to each kit ligand is calculated. For each embedding, theses distances are normalized to the [0,1] interval as described above and then the distances are subtracted from 1 as also described above. The resulting distances are such that 1 corresponds to the closest ligand (identity of the kit ligand) and 0 is the ligand that is farthest away in the kit ligand. In the end, the mean over all distances with respect to the different embeddings is taken. This results in a distance matrix of N×M, where N is the number of ligands for which recommendations are made and M is the number of kit ligands (M=24).

The performance, (here yield/conversion) is also normalized to [0,1] and converted into weights by an exponential function of the form 2^{x-1} where x is the performance, resulting in 24 weights for the 24 kit ligands. These weights are then column-wise multiplied with the distance matrix and then the maximum along the ligand axis is taken, resulting in a score from 0 to 1 for each ligand for the best distance/performance combination with respect to the kit ligands.

The final ranking of the N ligands is then used to suggest new experiments by taking the top scoring ligands from the above procedure. In principle, any list of N ligands can be taken for which one can obtain or compute features. In our case, a list of roughly 400 monodentate phosphine ligands was used that we also based the initial clustering on for the creation of the ligand kit.

### Cross Coupling Screening Reactions

### Example 1

### General Procedure for Buchwald-Hartwig C—N Cross Coupling Reaction 1:

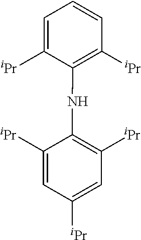 [SMILES: CC(C)c1cc(C(C)C)c(Nc2c(C(C)C)cccc2C(C)C)c(C(C)C)c1]

N-[2,6-Bis(1-methylethyl)phenyl]-2,4,6-tris(1-methylethyl)benzenamine (1): A 4 mL screw-capped vial was placed in a glovebox where Pd₂(dba)₃ (4.6 mg, 0.5 mmol, 0.5 mol %), phosphine ligand (0.1 mmol, 1.0 mol %), 4,4′-di-tert-butylbiphenyl (internal standard, 80 mg, 0.3 mmol, 0.3 equiv.), NaO-t-Bu (144 mg, 1.5 mmol, 1.5 equiv.), 2,4,6-triisopropylbromobenzene (0.25 mL, 1.0 mmol, 1.0 equiv.), 2,6-diisopropylanaline (0.23 mL, 1.2 mmol, 1.2 equiv.), and toluene (2.0 mL, 0.5 M) were added. The vial was sealed with a rubber/Teflon septum and taken out of the glovebox and the reaction was placed in a Radley Mya4 reaction station preheated to 80° C. and stirring set to 300 rpm. After a reaction time of either 1 h or 20 h, 50 μL of the sample was diluted with 1 mL EtOAc through a syringe filter and the reaction mixture was analyzed by GC. After the reaction had reached completion as judged by GC, the reaction mixture was diluted with ethyl acetate and filtered through a short plug of silica gel. After drying, the crude reaction mixture was dry loaded onto Biotage flash chromatography on silica gel (0-20% EtOAc/heptane) to obtain N-[2,6-bis(1-methylethyl)phenyl]-2,4,6-tris(1-methylethyl)benzenamine as a colorless solid. The product was confirmed by comparison with literature NMR spectral data (Raders, S. M.; Moore, J. N.; Parks, J. K.; Miller, A. D.; Leißing, T. M.; Kelley, S. P.; Rogers, R. D.; Shaughnessy, K. H. Trineopentylphosphine: A Conformationally Flexible Ligand for the Coupling of Sterically Demanding Substrates in the Buchwald-Hartwig Amination and Suzuki-Miyaura Reaction. J. Org. Chem. 2013, 78, 4649-4664).

¹H NMR (CDCl₃): δ 7.7 (d, J=7.6 Hz, 2H), 6.98-6.93 (m, 3H), 4.77 (s, 1H), 3.16-3.2 (m, 4H), 3.7 (sept, J=6.7 Hz, 1H), 1.24 (d, J=6.9 Hz, 6H), 1.8 (t, J=7.1 Hz, 24H).

¹³C NMR (CDCl₃): θ 143.8, 141.9, 141.2, 140.1, 138.3, 124.0, 122.3, 121.8, 34.2, 28.1, 27.9, 24.5, 23.9, 23.8.

### Kit Ligand Screening for Buchwald-Hartwig C—N Cross Coupling Reaction 1^{a}

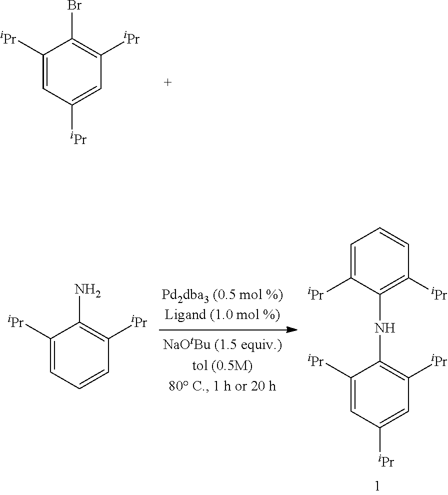 [SMILES: CC(C)c1cc(C(C)C)c(Br)c(C(C)C)c1 ; CC(C)c1cccc(C(C)C)c1N ; CC(C)c1cc(C(C)C)c(Nc2c(C(C)C)cccc2C(C)C)c(C(C)C)c1]

TABLE 2
Kit Ligand Screening for Buchwald-Hartwig
C—N Cross Coupling Reaction 1^{a}.

% Conversion^{b}

| Ligand | 1 h | 20 h |
|---|---|---|
| L1 | 0.00 | 2.96 |
| L2 | 0.86 | 7.55 |
| L3 | 0.95 | 9.28 |
| L4 | 27.76 | 53.88 |
| L5 | 51.82 | 97.44 |
| L6 | 24.97 | 53.47 |
| L7 | 2.27 | 10.73 |
| L8 | 11.98 | 51.39 |
| L9 | 19.28 | 95.8 |
| L10 | 8.76 | 48.38 |
| L11 | 0.00 | 0.00 |
| L12 | 0.00 | 0.58 |
| L13 | 0.00 | 3.35 |
| L14 | 3.24 | 18.99 |
| L15 | 8.00 | 39.73 |
| L16 | 16.52 | 45.84 |
| L17 | 24.60 | 47.42 |
| L18 | 12.84 | 25.14 |
| L19 | 0.00 | 0.5 |
| L20 | 3.27 | 18.35 |
| L21 | 6.15 | 29.25 |
| L22 | 2.88 | 12.80 |
| L23 | 0.00 | 3.00 |
| L24 | 16.8 | 47.8 |

^{a}Conditions: Ar—Br (1.0 mmol), Ar—NH₂ (1.2 mmol), Pd₂dba₃ (0.5 mol %), Ligand (1.0 mol %), NaO^{t}Bu (1.5 mmol), toluene (0.5M), 80° C., 1 or 20 h.
^{b}Average of 2 runs, % conversion determined by GC.

### Predicted Ligand Structures for Buchwald-Hartwig C—N Cross Coupling Reaction 1

TABLE S3
Predicted Ligand Structures for Buchwald-Hartwig C—N Cross Coupling
Reaction 1.

| Predicted Ligand # | Predicted Ligand Name | Ligand Structure |
|---|---|---|
| PL1 | VPhos | 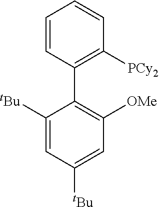 |
| PL2 | Bis(3,5- bis(trifluoromethyl)phenyl) (2′,6′-bis(dimethylamino)-3,6- dimethoxybiphenyl-2- yl)phosphine | 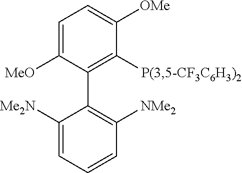 |
| PL3 | Tri(m-tolyl)phosphine | 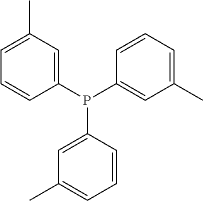 [SMILES: Cc1cccc(P(c2cccc(C)c2)c2cccc(C)c2)c1] |
| PL4 | RuPhos | 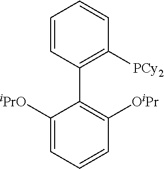 |
| PL5 | Diphenyl(p-tolyl) phosphine | 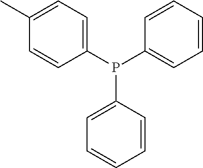 [SMILES: Cc1ccc(P(c2ccccc2)c2ccccc2)cc1] |
| PL6 | Tris(3,5-dimethylphenyl) phosphine | 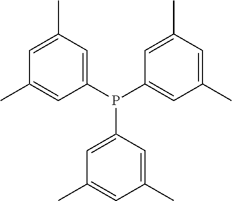 |
| PL7 | 4-(Diphenylphosphino) styrene | 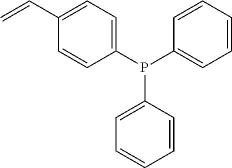 [SMILES: C=Cc1ccc(P(c2ccccc2)c2ccccc2)cc1] |
| PL8 | JackiePhos | 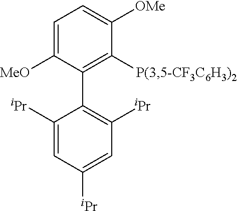 |
| PL9 | 9-[2- (Dicyclohexylphosphino) phenyl]-9H-carbazole | 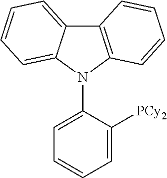 |
| PL10 | Triphenylphosphine | 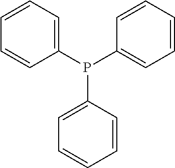 [SMILES: c1ccc(P(c2ccccc2)c2ccccc2)cc1] |

### Predicted Ligand Screening Results for Buchwald-Hartwig C—N Cross Coupling Reaction 1^{a}

TABLE S4
Predicted Ligand Screening Results for Buchwald-
Hartwig C—N Cross Coupling Reaction 1^{a}.

% Conversion^{b}

| Ligand | T = 1 h | T = 20 h |
|---|---|---|
| PL1 | 4.20 | 6.25 |
| PL2 | 23.82 | 87.43 |
| PL3 | 1.54 | 21.17 |
| PL4 | 1.86 | 95.18 |
| PL5 | 28.94 | 100 |
| PL6 | 8.35 | 100 |
| PL7 | 4.55 | 44.13 |
| PL8 | 15.40 | 100 |
| PL9 | 12.9 | 62.32 |
| PL10 | 71.67 | 100 |

^{a}Conditions: Ar—Br (1.0 mmol), Ar—NH₂ (1.2 mmol), Pd₂dba₃ (0.5 mol %), Ligand (1.0 mol %), NaO^{t}Bu (1.5 mmol), toluene (0.5M), 80° C., 1 or 20 h.
^{b}GC conversion.

### Example 2

### General Procedure for Buchwald-Hartwig C—N Cross Coupling Reaction 2:

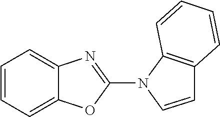 [SMILES: c1ccc2c(c1)ccn2-c1nc2ccccc2o1]

2-(1H-Indol-1-yl)benzoxazole (2): A 4 mL screw-capped vial was placed in a glovebox where Pd₂(dba)₃ (4.6 mg, 0.5 mmol, 0.5 mol %), phosphine ligand (0.1 mmol, 1.0 mol %), 4,4′-di-tert-butylbiphenyl (internal standard, 80 mg, 0.3 mmol, 0.3 equiv.), NaO-t-Bu (144 mg, 1.5 mmol, 1.5 equiv.), indole (141 mg, 1.2 mmol, 1.2 equiv.), K₃PO₄ (318 mg, 1.5 mmol, 1.5 equiv.), toluene (2.0 mL, 0.5 M), and 2-chlorobenzoxazole (114 μL, 1.0 mmol, 1.0 equiv.) were added. The vial was sealed with a rubber/Teflon septum and taken out of the glovebox and the reaction was placed in a Radley Mya4 reaction station preheated to 80° C. and stirring set to 300 rpm. After 16 h, 50 μL of the sample was diluted with 1 mL EtOAc through a syringe filter and the reaction mixture was analyzed by GC. After the reaction had reached completion as judged by GC, the reaction mixture was diluted with ethyl acetate and filtered through a short plug of silica gel. After drying, the crude reaction mixture was dry loaded onto Biotage flash chromatography on silica gel (0-5% EtOAc/heptane) to obtain 2-(1H-indol-1-yl)benzoxazole as a colorless solid. The product was confirmed by comparison with literature NMR spectral data (Li, D.-H.; Lan, X.-B.; Song, A.-X. Rahman, M. M.; Xu, C.; Huang, F.-D.; Szostak, R.; Szostak, M.; Liu, F.-S. Buchwald-Hartwig Amination of Coordinating Heterocycles Enabled by Large-but-Flexible Pd-BIAN-NHC Catalysts. Chem. Eur. J. 2022, 28, e202103341).

¹H NMR (CDCl₃) θ 8.57 (d, J=8.3 Hz, 1H), 7.88 (d, J=3.6 Hz, 1H), 7.73-7.64 (m, 2H), 7.55 (d, J=7.8 Hz, 1H), 7.49-7.41 (m, 1H), 7.33 (m, 3H), 6.79 (d, J=3.6 Hz, 1H).

¹³C NMR (CDCl₃) δ 154.8, 148.5, 141.5, 134.7, 130.2, 124.9, 124.7, 124.6, 123.6, 123.0, 121.3, 118.9, 114.6, 109.9, 108.5.

### Kit Ligand Screening for Buchwald-Hartwig C—N Cross Coupling Reaction 2^{a}

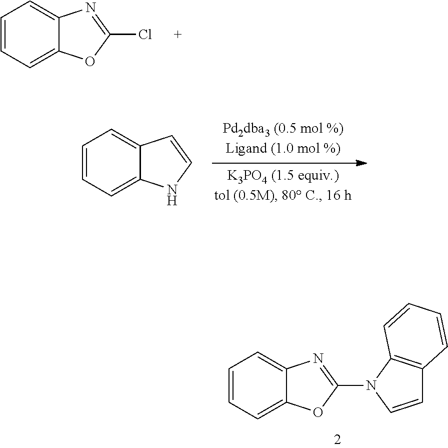 [SMILES: Clc1nc2ccccc2o1 ; c1ccc2[nH]ccc2c1 ; c1ccc2c(c1)ccn2-c1nc2ccccc2o1]

TABLE S5
Kit Ligand Screening for Buchwald-Hartwig
C—N Cross Coupling Reaction 2^{a}.

| Ligand | % Conversion^{b} |
|---|---|
| L1 | 6.17 |
| L2 | 5.16 |
| L3 | 18.61 |
| L4 | 7.48 |
| L5 | 4.50 |
| L6 | 6.91 |
| L7 | 6.40 |
| L8 | 6.44 |
| L9 | 4.61 |
| L10 | 5.99 |
| L11 | 4.86 |
| L12 | 22.56 |
| L13 | 18.46 |
| L14 | 12.47 |
| L15 | 7.22 |
| L16 | 3.45 |
| L17 | 25.91 |
| L18 | 10.38 |
| L19 | 4.82 |
| L20 | 5.79 |
| L21 | 9.56 |
| L22 | 11.45 |
| L23 | 5.74 |
| L24 | 7.80 |

^{a}Conditions: Ar—Cl (1.0 mmol), indole (1.2 mmol), Pd₂dba₃ (0.5 mol %), Ligand (1.0 mol %), K₃PO₄ (1.5 mmol), toluene (0.5M), 80° C., 16 h.
^{b}Average of 2 runs, % conversion determined by GC.

### Predicted Ligand Structures for Buchwald-Hartwig C—N Cross Coupling Reaction 2

TABLE S6
Predicted Ligand Structures for Buchwald-Hartwig C—N Cross Coupling
Reaction 2.

| Predicted | | |
|---|---|---|
| Ligand # | Predicted Ligand Name | Ligand Structure |
| PL11 | VPhos | 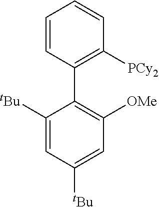 |
| PL12 | tBuMePhos | 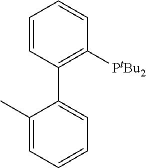 |
| PL13 | CPhos | 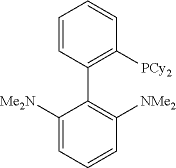 |
| PL14 | RuPhos | 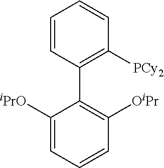 |
| PL15 | Dicyclohexyl(2′- methoxy[1,1′-biphenyl]-2-yl) phosphine | 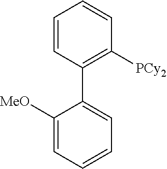 |
| PL16 | Bis(diethylamino)phenyl phosphine | 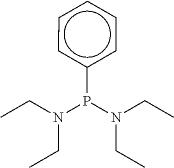 |
| PL17 | RockPhos | 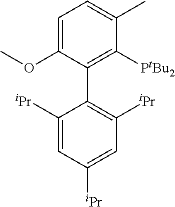 |
| PL18 | Methyl N,N,N′,N′- tetraisopropyl phosphorodiamidite | 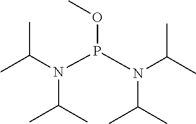 [SMILES: COP(N(C(C)C)C(C)C)N(C(C)C)C(C)C] |
| PL19 | Bis(3,5- bis(trifluoromethyl)phenyl) (2′,6′-bis(dimethylamino)-3,6- dimethoxybiphenyl-2- yl)phosphine | 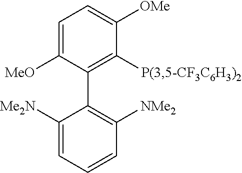 |
| PL20 | MePhos | 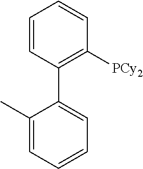 |

### Predicted Ligand Screening Results for Buchwald-Hartwig C—N Cross Coupling Reaction 2^{a}

TABLE S7
Predicted Ligand Screening Results for Buchwald-
Hartwig C—N Cross Coupling Reaction 2^{a}.

| Ligand | % Conversion^{b} |
|---|---|
| PL11 | 12.63 |
| PL12 | 11.51 |
| PL13 | 16.43 |
| PL14 | 21.40 |
| PL15 | 15.24 |
| PL16 | 13.67 |
| PL17 | 10.37 |
| PL18 | 5.26 |
| PL19 | 12.43 |
| PL20 | 11.37 |

^{a}Conditions: Ar—Cl (1.0 mmol), indole (1.2 mmol), Pd₂dba₃ (0.5 mol %), Ligand (1.0 mol %), K₃PO₄ (1.5 mmol), toluene (0.5M), 80° C., 16 h.
^{b}GC conversion.

Optimization of Reaction 2 with RuPhos^{a}

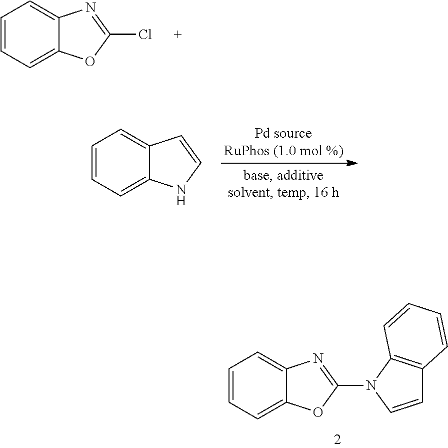 [SMILES: Clc1nc2ccccc2o1 ; c1ccc2[nH]ccc2c1 ; c1ccc2c(c1)ccn2-c1nc2ccccc2o1]

TABLE S8
Optimization of Reaction 2 with RuPhos^{a}.

| Entry | Solvent | Temp (° C.) | Base (equiv.) | % Conversion |
|---|---|---|---|---|
| STD | Tol | 80 | K₃PO₄ (1.5) | 21.40 |
| 1 | Tol | 80 | NaO^{t}Bu (1.5) | 28.91 |
| 2 | Tol | 80 | K₃PO₄ (3.0) | 31.34 |
| 3 | Tol | 100 | K₃PO₄ (3.0) | 43.25 |
| 4 | 1,4-Dioxane | 100 | K₃PO₄ (3.0) | 53.32 |
| 5 | THF | 80 | K₃PO₄ (3.0) | 50.49 |
| 6 | 1,4-Dioxane | 100 | K₃PO₄ (3.0) | 52.4 |
| 7^{c} | 1,4-Dioxane | 100 | K₃PO₄ (3.0) | 61.12 |
| 8^{d} | 1,4-Dioxane | 100 | K₃PO₄ (3.0) | 58.49 |
| 9^{c,d} | 1,4-Dioxane | 100 | K₃PO₄ (3.0) | 68.92 |

^{a}Standard conditions: Ar—Cl (1.0 mmol), indole (1.2 mmol), Pd₂dba₃ (0.5 mol %), RuPhos (1.0 mol %), base (1.5 mmol), solvent (0.5M), 16 h.
^{b}GC conversion.
^{c}Indole (4 equiv.).
^{d}ACN (1 equiv.).

### Example 3

### General Procedure for Suzuki C—C Cross Coupling Reaction 3:

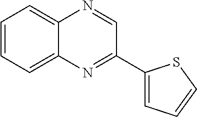 [SMILES: c1csc(-c2cnc3ccccc3n2)c1]

2-(2-Thienyl)quinoxaline (3): A 4 mL screw-capped vial was placed in a glovebox where Pd₂(dba)₃ (4.6 mg, 0.5 mmol, 0.5 mol %), phosphine ligand (0.1 mmol, 1.0 mol %), 4,4′-di-tert-butylbiphenyl (internal standard, 80 mg, 0.3 mmol, 0.3 equiv.), 2-thienylboronic acid (192 mg, 1.5 mmol, 1.5 equiv.), 2-chloroquinoxaline (165 mg, 1.0 mmol, 1.0 equiv.), toluene (2.0 mL, 0.5 M), and Et₃N (420 μL, 3.0 mmol, 3.0 equiv.) were added. The vial was sealed with a rubber/Teflon septum and taken out of the glovebox and the reaction was placed in a Radley Mya4 reaction station preheated to 100° C. and stirring set to 300 rpm. After 20 h, 50 μL of the sample was diluted with 1 mL EtOAc through a syringe filter and the reaction mixture was analyzed by GC. After the reaction had reached completion as judged by GC, the reaction mixture was diluted with ethyl acetate and filtered through a short plug of silica gel. After drying, the crude reaction mixture was dry loaded onto Biotage flash chromatography on silica gel (0-30% EtOAc/heptane) to obtain 2-(2-thienyl)quinoxaline as a white solid. The product was confirmed by comparison with literature NMR spectral data (Knapp, D. M.; Gillis, E. P.; Burke, M. D. A General Solution for Unstable Boronic Acids: Slow-Release Cross-Coupling from Air-Stable MIDA Boronates. J. Am. Chem. Soc. 2009, 131, 20, 6961-6963).

¹H NMR (500.1 MHz, CDCl₃): δ 9.25 (s, 1H), 8.9-8.7 (m, 2H), 7.88-7.87 (m, 1H), 7.78-7.69 (m, 2H), 7.56-7.55 (m, 1H), 7.23-7.21 (m, 1H).

¹³C NMR (CDCl₃): δ 147.3, 142.2, 142.1, 142.0, 141.3, 130.4, 129.7, 129.1, 129.1, 128.4, 126.

### Kit Ligand Screening for Suzuki C—C Cross Coupling Reaction 3^{a}

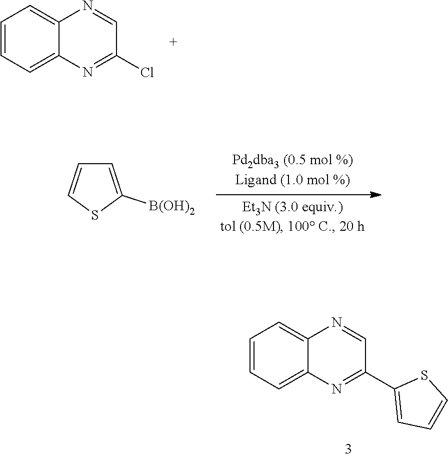 [SMILES: Clc1cnc2ccccc2n1 ; OB(O)c1cccs1 ; c1csc(-c2cnc3ccccc3n2)c1]

TABLE S9
Kit Ligand Screening for Suzuki
C—C Cross Coupling Reaction 3^{a}.

| Ligand | % Conversion^{b} |
|---|---|
| L1 | 0.00 |
| L2 | 5.80 |
| L3 | 4.4 |
| L4 | 26.33 |
| L5 | 21.77 |
| L6 | 3.76 |
| L7 | 0.00 |
| L8 | 28.76 |
| L9 | 2.48 |
| L10 | 2.35 |
| L11 | 16.00 |
| L12 | 27.19 |
| L13 | 31.70 |
| L14 | 1.85 |
| L15 | 15.21 |
| L16 | 26.9 |
| L17 | 52.42 |
| L18 | 3.56 |
| L19 | 3.51 |
| L20 | 3.68 |
| L21 | 45.21 |
| L22 | 27.87 |
| L23 | 3.37 |
| L24 | 9.68 |

^{a}Conditions: Ar—Cl (1.0 mmol), Ar—B(OH)₂ (1.5 mmol), Pd₂dba₃ (0.5 mol %), Ligand (1.0 mol %), Et₃N (3.0 mmol), toluene (0.5M), 100° C., 20 h.
^{b}Average of 2 runs, % conversion determined by GC.

### Predicted Ligand Structures for Suzuki C—C Cross Coupling Reaction 3

TABLE S10
Predicted Ligand Structures for Suzuki C—C Cross Coupling Reaction 3.

| Predicted | | |
|---|---|---|
| Ligand # | Predicted Ligand Name | Ligand Structure |
| PL21 | VPhos | 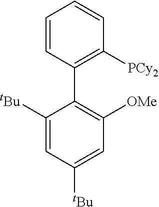 |
| PL22 | 2-Diphenylphosphino- 6-methylpyridine | 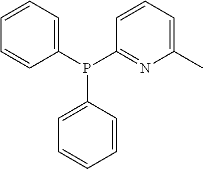 [SMILES: Cc1cccc(P(c2ccccc2)c2ccccc2)n1] |
| PL23 | Bis(3,5- bis(trifluoromethyl)phenyl) (2′,6′-bis(dimethylamino)-3,6- dimethoxybiphenyl-2- yl)phosphine | 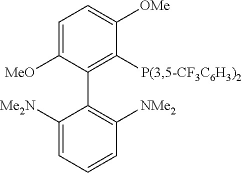 |
| PL24 | CPhos | 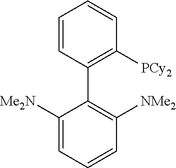 |
| PL25 | RuPhos | 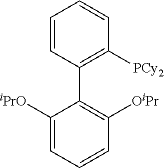 |
| PL26 | Dicyclohexyl(2′-methoxy[1,1′- biphenyl]-2-yl)phosphine | 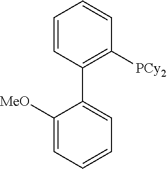 |
| PL27 | 2′-Dicyclohexylphosphino- 2-methoxy-1- phenylnaphthalene | 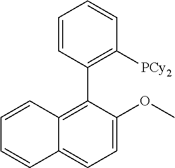 |
| PL28 | 3-(Diphenylphosphino)phenol | 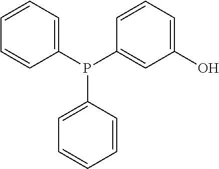 [SMILES: Oc1cccc(P(c2ccccc2)c2ccccc2)c1] |
| PL29 | MePhos | 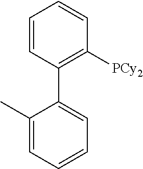 |
| PL30 | cBRIDP | 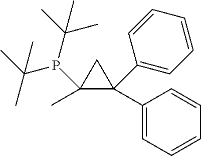 [SMILES: CC(C)(C)P(C(C)(C)C)C1(C)CC1(c1ccccc1)c1ccccc1] |
| PL31 | Di-tert-butyl(2′,6′-dimethoxy- [1,1′-biphenyl]-2-yl)phosphine | 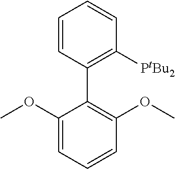 |
| PL32 | 9-[2-(Dicyclohexylphosphino) phenyl]-9H-carbazole | 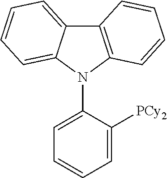 |
| PL33 | TrixiePhos | 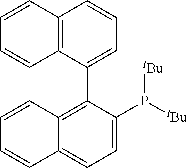 [SMILES: CC(C)(C)P(c1ccc2ccccc2c1-c1cccc2ccccc12)C(C)(C)C] |
| PL34 | JackiePhos | 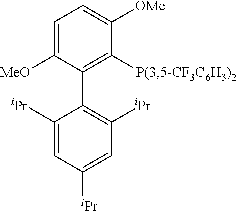 |

### Predicted Ligand Screening Results for Suzuki C—C Cross Coupling Reaction 3^{a}

TABLE S11
Predicted Ligand Screening Results for
Suzuki C—C Cross Coupling Reaction 3^{a}.

| Ligand | % Conversion^{b} |
|---|---|
| PL21 | 49.96 |
| PL22 | 2.4 |
| PL23 | 24.67 |
| PL24 | 37.82 |
| PL25 | 49.75 |
| PL26 | 43.36 |
| PL27 | 14.69 |
| PL28 | 45.67 |
| PL29 | 42.97 |
| PL30 | 11.63 |
| PL31 | 0 |
| PL32 | 31.61 |
| PL33 | 16.9 |
| PL34 | 20.39 |

^{a}Conditions: Ar—Cl (1.0 mmol), Ar—B(OH)₂ (1.5 mmol), Pd₂dba₃ (0.5 mol %), Ligand (1.0 mol %), Et₃N (3.0 mmol), toluene (0.5M), 100° C., 20 h.
^{b}GC conversion.

Suzuki C—C Cross Coupling Reaction 3 Optimization with RuPhos^{a}

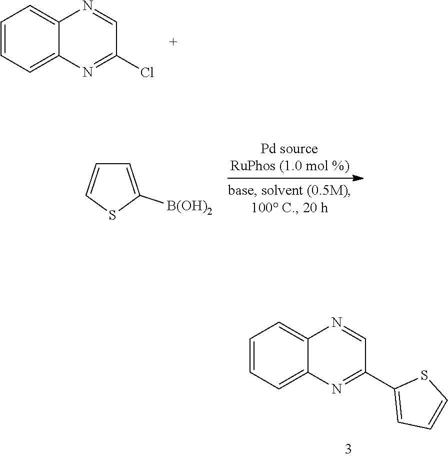 [SMILES: Clc1cnc2ccccc2n1 ; OB(O)c1cccs1 ; c1csc(-c2cnc3ccccc3n2)c1]

TABLE S12
Suzuki C—C Cross Coupling Reaction
3 Optimization with RuPhos^{a}.

| Entry | Solvent | Base (equiv.) | % Conversion^{b} |
|---|---|---|---|
| STD | Tol | Et₃N (3.0) | 49.75 |
| 1 | Tol | K₃PO₄ (3.0) | 33.89 |
| 2 | Tol | Cs₂CO₃ (3.0) | 4.47 |
| 3 | 1,4-Dioxane | Et₃N (3.0) | 28.17 |
| 4 | ^{t}BuOH | Et₃N (3.0) | 55.36 |
| 5^{c} | 10:1 Tol:H₂O | Et₃N (3.0) | 55.11 |
| 6^{d} | ^{t}BuOH | Et₃N (3.0) | 50.89 |

^{a}Standard conditions: Ar—Cl (1.0 mmol), Ar—B(OH)₂ (1.5 mmol), Pd₂dba₃ (0.5 mol %), RuPhos (1.0 mol %), base (3.0 mmol), solvent (0.5M), 16 h.
^{b}GC conversion.
^{c}Reaction temperature = 80° C.
^{d}Ar—B(OH)2 (4 equiv.).

Each of the characteristics and examples described above, and combination thereof, may be said to be encompassed by the present disclosure. The present disclosure is thus drawn to the following non-limiting aspects:
(1) A method for optimizing chemical reactions utilizing machine learning, the method comprising: selecting a chemical space for grouping, each chemical space defined by a plurality of prospective chemicals; selecting a plurality of chemical features, each chemical feature corresponding to the plurality of prospective chemicals, grouping the plurality of prospective chemicals in a grouping space based upon the plurality of chemical features; selecting a plurality of representative chemicals from the prospective chemicals, each representative chemical corresponding to a group of the plurality of prospective chemicals as grouped within the grouping space; and assembling a test kit having a plurality of test chemicals, each test chemical corresponding to a representative chemical of the plurality of representative chemicals.
(2) The method according to aspect 1, wherein the grouping space is defined by the plurality of chemical features.
(3) The method according to aspect 1, wherein the grouping space is a dimensionally reduced space of the plurality of chemical features.
(4) The method according to aspect 1, the method further comprising: generating a plurality of reduced chemical spaces, wherein each reduced chemical spaces is a dimensionally reduced space of the chemical space.
(5) The method according to aspect 4, the method further comprising: calculating a plurality of distances, wherein each distance of the plurality of distances is a distance between a prospective chemical of the plurality of prospective chemicals and a test chemical of the plurality of test chemicals.
(6) The method according to aspect 5, wherein the plurality of test chemicals is a subset of the plurality of prospective chemicals.
(7) The method according to aspect 5, wherein the plurality of prospective chemicals does not include the plurality of test chemicals.
(8) The method according to aspect 4, the method further comprising: calculating a plurality of distance metrics, wherein each distance metric of the plurality of distance metrics is one minus a distance between one of the plurality of prospective chemicals and one of the plurality of test chemicals.
(9) The method according to aspect 8, the method further comprising averaging each of the plurality of distance metrics across all of the reduced chemical spaces to generate a plurality of averaged distance metrics.
(10) The method according to aspect 1, the method further comprising generating a plurality of weights, wherein each weight of the plurality of weights corresponds to one of a plurality of results, wherein each result of the plurality of results corresponds to one of the plurality of test chemicals.
(11) The method according to aspect 10, wherein the plurality of weights are determined in accordance with an exponential function.
(12) The method according to aspect 10, wherein each of a plurality of distances of distance metrics is multiplied by a respective weight of the plurality of weights.
(13) The method according to aspect 12, wherein a maximum value for each respective prospective chemical is taken across all of the multiplied plurality of distance metrics between the respective prospective chemical and the plurality of test chemicals.
(14) The method according to aspect 13, the method further comprising ranking the maximum value for each respective prospective chemical.
(15) The method according to aspect 1, the method further comprising testing the plurality of test chemicals to determine a plurality of results, wherein each of the plurality of results corresponds to a respective result of a test chemical of the plurality of test chemicals.
(16) The method according to aspect 15, wherein the plurality of results is uploaded into a server.
(17) The method according to aspect 15, the method further comprising selecting a prediction space utilizing a plurality of predictive features to provide a prediction to a result space, wherein the plurality of results define data points within the prediction space mapped to the result space.
(18) The method according to aspect 17, wherein the result space includes a chemical yield.
(19) The method according to aspect 17, wherein the result space is a side-product metric.
(20) The method according to aspect 17, wherein the result space is a single parameter.
(21) The method according to aspect 17, wherein the prediction space includes the grouping space.
(22) The method according to aspect 17, wherein the prediction space includes the plurality of chemical features therein.
(23) The method according to aspect 17, wherein a computer is configured to fit the plurality of results using a regression fit to map the plurality of predictive features to the result space.
(24) The method according to aspect 21, the method further comprising selecting a chemical from the plurality of prospective chemicals corresponding to an optimized value of the result space.
(25) The method according to aspect 17, where a computer is configured to train a learner using the plurality of results.
(26) The method according to aspect 25, wherein the learner is at least one of an artificial neural network, a K-nearest neighbor, a decision tree, a random forest, a support vector machine, a Bayesian regressor, and an ensemble.
(27) The method according to aspect 1, wherein the plurality of chemical features include a plurality of chemical featurizations.
(28) The method according to aspect 1, wherein the plurality of chemical features include a plurality of molecular descriptors.
(29) The method according to aspect 1, wherein the chemical space includes a plurality of catalysts.
(30) The method according to aspect 1, wherein the chemical space includes a plurality of ligands.
(31) The method according to aspect 1, wherein the plurality of chemical features are determined via at least one of cheminformatics and computational modelling on the computer.
(32) The method according to aspect 1, wherein the plurality of chemical features is stored on a database executed by a computer.
(33) The method according to aspect 1, the method further comprising: testing the plurality of test chemicals to determine a plurality of results, wherein each of the plurality of results corresponds to a respective result of a test chemical of the plurality of test chemicals; selecting a prediction space utilizing a plurality of predictive features to provide a prediction to a result space, wherein the plurality of results define data points within the prediction space; mapping the plurality of predictive features to the result space using regression; determining a best catalyst or best ligand of all available catalysts or ligands for a catalytic chemical reaction in the prediction space in accordance with a prediction; and performing the catalytic chemical reaction with the best catalyst or best ligand in accordance with the prediction.
(34) The method according to aspect 1, the method wherein the grouping space is generated by performing dimensionality reduction on the plurality of chemical features.
(35) The method according to aspect 34, wherein the dimensionality reduction is implemented on a computer utilizing principal component analysis.
(36) The method according to aspect 1, wherein the chemical space is defined by a plurality of phosphine ligands for cross-coupling reactions.
37) The method according to aspect 36, wherein the cross-coupling reactions include one of a Suzuki catalysis and a Buchwald catalysis.
(38) The method according to aspect 1, wherein the plurality of test chemicals consists of 24 chemicals.
(39) The method according to aspect 1, wherein the plurality of chemical features includes at least one of a space-filling feature, a bulk feature, an orientation feature, an electrical feature, a Vin, a frontier Mos, a Fukui function, an NBO analyses, a NMR tensor, a steric property, a Sterimol L, B1, B5, B1, B5, a quadrant analysis, an octant analysis, a total volume, a buried volume, a dipole moment, an energy of solvation, and a dispersion of potential.
(40) The method according to aspect 17, wherein the plurality of predictive features includes at least one of a space-filling feature, a bulk feature, an orientation feature, an electrical feature, a Vin, a frontier Mos, a Fukui function, an NBO analyses, a NMR tensor, a steric property, a Sterimol L, B1, B5, B1, B5, a quadrant analysis, an octant analysis, a total volume, a buried volume, a dipole moment, an energy of solvation, and a dispersion of potential.
(41) A method for utilizing an experimental test kit, the method comprising: assembling a test kit having a plurality of test chemicals, each test chemical corresponding to a representative chemical of a plurality of representative chemicals; testing the plurality of test chemicals to determine a plurality of results, wherein each of the plurality of results corresponds to a respective result of a respective test chemical of the plurality of test chemicals; and determining a best catalyst or best ligand of all available catalysts or ligands for a catalytic chemical reaction in a prediction space in accordance with a prediction.
(42) The method according to aspect 41, the method further comprising: generating a plurality of reduced chemical spaces, wherein each reduced chemical spaces is a dimensionally reduced space of a chemical space.
(43) The method according to aspect 42, the method further comprising: calculating a plurality of distances, wherein each distance of the plurality of distances is a distance between a prospective chemical of a plurality of prospective chemicals and a test chemical of the plurality of test chemicals.
(44) The method according to aspect 43, further comprising: calculating a plurality of distance metrics, wherein each distance metric of the plurality of distance metrics is one minus a distance between one of the plurality of prospective chemicals and one of the plurality of test chemicals.
(45) The method according to aspect 44, further comprising averaging each of the plurality of distance metrics across all of the reduced chemical spaces to generate a plurality of averaged distance metrics.
(46) The method according to aspect 41, further comprising generating a plurality of weights, wherein each weight of the plurality of weights corresponds to one of a plurality of results, wherein each result of the plurality of results corresponds to one of the plurality of test chemicals.
(47) The method according to aspect 46, wherein the plurality of weights are determined in accordance with an exponential function.
(48) The method according to aspect 46, wherein each of a plurality of distances of distance metrics is multiplied by a respective weight of the plurality of weights.
(49) The method according to aspect 48, wherein a maximum value for each respective prospective chemical is taken across all of the multiplied plurality of distance metrics between the respective prospective chemical and the plurality of test chemicals.
(50) The method according to aspect 49, further comprising ranking the maximum value for each respective prospective chemical to thereby find the best catalyst or best ligand.
(51) The method according to aspect 41, further comprising selecting the prediction space utilizing a plurality of predictive features to provide a prediction to a result space, wherein the plurality of results define data points within the prediction space.
(52) The method according to aspect 51, further comprising fitting the data points within the plurality of predictive features to the result space using regression.
(53) The method according to aspect 51, wherein the plurality of predictive features includes at least one of a space-filling feature, a bulk feature, an orientation feature, an electrical feature, a Vin, a frontier Mos, a Fukui function, an NBO analyses, a NMR tensor, a steric property, a Sterimol L, B1, B5, B1, B5, a quadrant analysis, an octant analysis, a total volume, a buried volume, a dipole moment, an energy of solvation, and a dispersion of potential.
(54) The method according to aspect 41, further comprising performing the catalytic chemical reaction with the best catalyst or best ligand in accordance with the prediction.
(55) The method according to aspect 33, wherein the plurality of results is uploaded into a server.
(56) The method according to aspect 33, further comprising selecting the prediction space utilizing a plurality of predictive features to provide a prediction to a result space, wherein the plurality of results defines data points within the prediction space.
(57) The method according to aspect 56, wherein a computer is configured map the plurality of predictive features to the result space using regression by fitting the plurality of results to the predictive features and the result space.
(58) A method to assemble a Test Kit for optimizing catalytic chemical reactions via a computer, the method comprising: parametrizing catalysts or ligands for a respective catalytic chemical reaction regarding respective chemical features which are specific for the catalytic chemical reaction via the computer; grouping the parametrized catalysts or ligands into a given number of clusters, which are spanning over a chemical space of the catalysts or ligands, based on their chemical features; using the computer to select one representative catalyst or ligand from each cluster according to predetermined criteria; and assembling the Test Kit with the selected representative catalysts or ligands as components.
(59) The method according to aspect 58, wherein the respective chemical features are determined via cheminformatics and computational modelling on the computer.
(60) The method according to aspect 58, further comprising performing the grouping act by using k-means clustering using the computer.
(61) The method according to aspect 58, wherein the selection is based on a combination of chemical features and commercial feasibility and/or sourcing availability of the catalysts or ligands as the predetermined criteria.
(62) The method according to aspect 61, wherein all available catalysts or ligands are stored on a database connected to the computer.
(63) A Test Kit with a specific given number of catalyst or ligand components assembled by using the method according to aspect 58.
(64) The method according to aspect 58, the method further comprising: performing standardized experiments for the catalytic chemical reactions with the components in the Test Kit; inputting result data of the performed experiments into the computer; using a machine learning regression model running on the computer to interpolate between the given number of clusters in the spanned chemical space of all available catalysts or ligands; using the machine learning regression model to predict the best fitting catalyst for the catalytic chemical reaction in the spanned chemical space of all available catalysts or ligands; and performing the catalytic chemical reaction with the predicted catalyst or ligand.
(65) The method according to aspect 64, wherein a web interface is provided via the computer via which a user uploads the results data of the chemical reactions from the performed experiments.
(66) A system for optimizing chemical reactions, the system implemented by an operative set of processor executable instructions configured for execution on at least one processor, the at least one processor and the operative set of processor executable instructions configured to: select a chemical space for grouping, each chemical space defined by a plurality of prospective chemicals; select a plurality of chemical features, each chemical feature corresponding to the plurality of prospective chemicals, group the plurality of prospective chemicals in a grouping space based upon the plurality of chemical features; select a plurality of representative chemicals from the prospective chemicals, each representative chemical corresponding to a group of the plurality of prospective chemicals as grouped within the grouping space; and recommend a test kit having a plurality of test chemicals, each test chemical corresponding to a representative chemical of the plurality of representative chemicals.
(67) A system implemented by an operative set of processor executable instructions configured for execution on at least one processor, the at least one processor and the operative set of processor executable instructions configured to: recommend a test kit having a plurality of test chemicals, each test chemical corresponding to a representative chemical of a plurality of representative chemicals; receive a plurality of results from a plurality of experiments performed using the plurality of test chemicals, wherein each of the plurality of results corresponds to a respective result of a respective test chemical of the plurality of test chemicals; and recommend a best catalyst or best ligand of all available catalysts or ligands for a catalytic chemical reaction in a prediction space in accordance with a prediction.
(68) A method for suggesting prospective chemicals that can be used to optimize chemical reactions, comprising: performing an initial screening experiment on a set of test chemicals to generate results; obtaining chemical features that describe properties of the prospective chemicals; reducing a dimensionality of the chemical features to generate a predetermined number of different chemical spaces; calculating distances between each of the predetermined number of chemical spaces and each of the test chemicals for a given prospective chemical; normalizing the distances for each of the predetermined number of chemical spaces to the [0,1] interval; subtracting the normalized distances from 1 to generate a distance metric for each prospective chemical and each test chemical in each chemical space; averaging the distance metrics over all chemical spaces for each prospective chemical; normalizing the results obtained from the initial screening experiment to [0,1] and converting them into weights; multiplying the weights with a distance matrix to obtain a weighted distance matrix, the distance matrix generated via the averaging act and having a prospective chemical axis and a test chemical axis; taking the maximum value along the prospective chemical axis to obtain a score for each prospective chemical; and ranking the N prospective chemicals from highest to lowest based on the obtained scores.
(69) The method of aspect 68, wherein the chemical features are DFT-based features.
(70) The method of aspect 68, wherein the chemical features are reduced using PCA, Space PCA, Kernel PCA with an RBF kernel, Kernel PCA with a cosine kernel, Fast ICA, Spectral Embedding, Isomap, or Local Linear Embedding.
(71) The method of aspect 68, wherein the results obtained from the initial screening experiment are yield or enantioselectivity.
(72) The method of aspect 68, wherein the weights obtained from the normalized results are column-wise multiplied with the distance matrix to obtain a weighted distance matrix.
(73) The method of aspect 68, wherein the chemical features are phosphine ligands' properties.
(74) A method for suggesting prospective chemicals for optimizing chemical reactions, comprising: obtaining a list of N prospective chemicals; obtaining chemical features configured to describe properties of the N prospective chemicals; clustering the N prospective chemicals based on their chemical features to obtain a set of representative chemicals, the set of representative chemicals define a set of test chemicals; calculating distances between each of the test chemicals and each of the representative chemicals using a distance metric based on the chemical features; normalizing the distances to the [0,1] interval; subtracting the normalized distances from 1 to generate a distance metric for each representative chemical and each test chemical; normalizing results obtained from an initial screening experiment to [0,1] and converting them into weights; multiplying the weights with a distance matrix to obtain a weighted distance matrix; taking the maximum value along a representative chemical axis to obtain a score for each representative chemical; and ranking the N prospective chemicals based on the scores obtained for their representative chemicals.
(75) A computer program product comprising a non-transitory computer-readable storage medium encoded with instructions for performing the steps of the method of aspect 74.
(76) A system for suggesting prospective chemicals that can be used to optimize chemical reactions, comprising a computer system configured to implement the method of aspect 74.
(77) The method according to aspect 74, wherein the distances are calculated after dimensionality reduction of the chemical features.
(78) A computer program product comprising a non-transitory computer-readable storage medium encoded with instructions for implementing the method of aspect 74.
(79) A system for identifying prospective chemicals for optimizing chemical reactions, comprising a computer system configured to implement the method of aspect 74.
(80) A method for suggesting prospective chemicals for optimizing chemical reactions, comprising: obtaining a list of N prospective chemicals; receiving results for M prospective chemicals thereby defining test chemicals; obtaining chemical features configured to describe properties of the N prospective chemicals; determining a score for each representative chemical; and ranking the N prospective chemicals based on the scores obtained for their representative chemicals.
(81) The method according to aspect 80, wherein the act of determining the score for each representative chemical comprises: calculating distances between each of the test chemicals and each of the representative chemicals using a distance metric for each of a plurality of dimensionally reduced spaces of the chemical features; normalizing the distances to the [0,1] interval for each of the plurality of dimensionally reduced spaces; subtracting the normalized distances from 1 to generate a distance metric for each representative chemical and each test chemical for each of the plurality of dimensionally reduced spaces; averaging the distance metric for each representative chemical and each test chemical across all of the dimensionally reduced spaces; normalizing the results obtained from the received results of the M prospective chemicals to [0,1] and converting them into weights; multiplying the weights with a distance matrix to obtain a weighted distance matrix, the distance matrix having a representative chemical axis and a test chemical axis; and taking the maximum value along a representative chemical axis to obtain a score for each representative chemical.
(82) The method according to aspect 81, wherein the test chemicals are removed from the representative chemical axis.
(83) A computer program product comprising a non-transitory computer-readable storage medium encoded with instructions for performing the steps of the method of aspect 80.
(84) A system for suggesting prospective chemicals that can be used to optimize chemical reactions, comprising a computer system configured to implement the method of aspect 80.

What is claimed is:

1. A method for optimizing chemical reactions utilizing machine learning, the method comprising:
selecting a chemical space for grouping, each chemical space defined by a plurality of prospective chemicals;
selecting a plurality of chemical features, each chemical feature corresponding to the plurality of prospective chemicals,
grouping the plurality of prospective chemicals in a grouping space based upon the plurality of chemical features;
selecting a plurality of representative chemicals from the prospective chemicals, each representative chemical corresponding to a group of the plurality of prospective chemicals as grouped within the grouping space; and
assembling a test kit having a plurality of test chemicals, each test chemical corresponding to a representative chemical of the plurality of representative chemicals.

2. The method according to claim 1, wherein the grouping space is defined by the plurality of chemical features.

3. The method according to claim 1, wherein the grouping space is a dimensionally reduced space of the plurality of chemical features.

4. The method according to claim 1, the method further comprising:
generating a plurality of reduced chemical spaces, wherein each reduced chemical spaces is a dimensionally reduced space of the chemical space.

5. The method according to claim 4, the method further comprising:
calculating a plurality of distances, wherein each distance of the plurality of distances is a distance between a prospective chemical of the plurality of prospective chemicals and a test chemical of the plurality of test chemicals.

6. The method according to claim 5, wherein the plurality of test chemicals is a subset of the plurality of prospective chemicals.

7. The method according to claim 5, wherein the plurality of prospective chemicals does not include the plurality of test chemicals.

8. The method according to claim 4, the method further comprising:
calculating a plurality of distance metrics, wherein each distance metric of the plurality of distance metrics is one minus a distance between one of the plurality of prospective chemicals and one of the plurality of test chemicals.

9. The method according to claim 8, the method further comprising averaging each of the plurality of distance metrics across all of the reduced chemical spaces to generate a plurality of averaged distance metrics.

10. The method according to claim 1, the method further comprising generating a plurality of weights, wherein each weight of the plurality of weights corresponds to one of a plurality of results, wherein each result of the plurality of results corresponds to one of the plurality of test chemicals.

11. The method according to claim 10, wherein the plurality of weights are determined in accordance with an exponential function.

12. The method according to claim 10, wherein each of a plurality of distances of distance metrics is multiplied by a respective weight of the plurality of weights.

13. The method according to claim 12, wherein a maximum value for each respective prospective chemical is taken across all of the multiplied plurality of distance metrics between the respective prospective chemical and the plurality of test chemicals.

14. The method according to claim 13, the method further comprising ranking the maximum value for each respective prospective chemical.

15. The method according to claim 1, the method further comprising testing the plurality of test chemicals to determine a plurality of results, wherein each of the plurality of results corresponds to a respective result of a test chemical of the plurality of test chemicals.

16. The method according to claim 15, wherein the plurality of results is uploaded into a server.

17. The method according to claim 15, the method further comprising selecting a prediction space utilizing a plurality of predictive features to provide a prediction to a result space, wherein the plurality of results define data points within the prediction space mapped to the result space.

18. The method according to claim 17, wherein the result space includes a chemical yield.

19. The method according to claim 17, wherein the result space is a side-product metric.

20. The method according to claim 17, wherein the result space is a single parameter.

21. The method according to claim 17, wherein the prediction space includes the grouping space.

22. The method according to claim 17, wherein the prediction space includes the plurality of chemical features therein.

23. The method according to claim 17, wherein a computer is configured to fit the plurality of results using a regression fit to map the plurality of predictive features to the result space.
